SEQUENCE LISTING (147 sequences)

SEQ ID NO: 1 - DNA, 5976 bp, Homo sapiens
accagagagtggatggcagaggtgggctgtagagccaaagtggggtgggagcgcgaagatggcagctgctgaggaggagccgaagcccaaaaagctgaaggtggaggcgccgcaagcgctgagagaaaatattctctttggaatgggaaatcctctgcttgacatctctgctgtagtggacaaagatttccttgataagtattctctgaaaccaaatgaccaaatcttggctgaagacaaacacaaggaactgtttgatgaacttgtgaaaaaattcaaagtcgaatatcatgctggtggctctacccagaattcaattaaagtggctcagtggatgattcaacagccacacaaagcagcaacattttttggatgcattgggatagataaatttggggagatcctgaagagaaaagctgctgaagcccatgtggatgctcattactacgagcagaatgagcagccaacaggaacttgtgctgcatgcatcactggtgacaacaggtccctcatagctaatcttgctgctgccaattgttataaaaaggaaaaacatcttgatctggagaaaaactggatgttggtagaaaaagcaagagtttgttatatagcaggcttttttcttacagtttccccagagtcagtattaaaggtggctcaccatgcttctgaaaacaacaggattttcactttgaatctatctgcaccgtttattagccagttctacaaggaatcattgatgaaagttatgccttatgttgatatactttttggaaatgagacagaagctgccacttttgctagagagcaaggctttgagactaaagacattaaagagatagccaaaaagacacaagccctgccaaagatgaactcaaagaggcagcgaatcgtgatcttcacccaagggagagatgacactataatggctacagaaagtgaagtcactgcttttgctgtcttggatcaagaccagaaagaaattattgataccaatggagctggagatgcatttgttggaggttttctgtctcaactggtctctgacaagcctctgactgaatgtatccgtgctggccactatgcagcaagcatcataattagacggactggctgcacctttcctgagaagccagacttccactgatggaagagctgaaaacacaagcccaggagtgcagacactgccctaattgcttcctgagaattcccatattaataaagaagaaaattatctgccattttttcctactataataatgctgaatcttaatttagagggtacaagggtatggtaatgcttgtagaatctttattatctcaacaatctaaaaaatgatgtttatttccatagtttgatagtgccacttaaatgccaattaaacaagaatataacatttcaatagaaatttttatttcattttcaattactttgtaaattcgtgtgtatttagtacactgatttgtttttttacatttctgctttgaatgcagatgcaatttaatataatagattttttaatgaattaatcttaacatagtaatctttagctttttatacaaatatatttaatttaggagtatatgtgtgtctatacacacacatacataaatataccacatatacacctgatagtcaaataaggtacagaaattttatcttgtcaattatgccaaataatctctttaatgtgcactcaaacatgtaataaactttggataattaaataatgtgccaaatttaagttgtaccacaatattcaaatcatagtcttatggaactctgtaattggacacaaacaaaaacttgtcatataggaatttttttctgtcttttccctgtgtacagcaaactgaggaagaaagatcattatttaatactgggccctgaaatgacacccaatttgttaatggaattatgaagaacagcaatataatacactgaagaataacttagtatcagagaccaataaatcacttttttaaggatgaaccagagagtggatggcagaggtgggctgtagagccaaagtggggtgggagcgcgaagatggcagctgctgaggaggagccgaagcccaaaaagctgaaggtggaggcgccgcaagcgctgagagaaaatattctctttggaatgggaaatcctctgcttgacatctctgctgtagtggacaaagatttccttgataagtattctctgaaaccaaatgaccaaatcttggctgaagacaaacacaaggaactgtttgatgaacttgtgaaaaaattcaaagtcgaatatcatgctggtggctctacccagaattcaattaaagtggctcagtggatgattcaacagccacacaaagcagcaacattttttggatgcattgggatagataaatttggggagatcctgaagagaaaagctgctgaagcccatgtggatgctcattactacgagcagaatgagcagccaacaggaacttgtgctgcatgcatcactggtgacaacaggtccctcatagctaatcttgctgctgccaattgttataaaaaggaaaaacatcttgatctggagaaaaactggatgttggtagaaaaagcaagagtttgttatatagcaggcttttttcttacagtttccccagagtcagtattaaaggtggctcaccatgcttctgaaaacaacaggattttcactttgaatctatctgcaccgtttattagccagttctacaaggaatcattgatgaaagttatgccttatgttgatatactttttggaaatgagacagaagctgccacttttgctagagagcaaggctttgagactaaagacattaaagagatagccaaaaagacacaagccctgccaaagatgaactcaaagaggcagcgaatcgtgatcttcacccaagggagagatgacactataatggctacagaaagtgaagtcactgcttttgctgtcttggatcaagaccagaaagaaattattgataccaatggagctggagatgcatttgttggaggttttctgtctcaactggtctctgacaagcctctgactgaatgtatccgtgctggccactatgcagcaagcatcataattagacggactggctgcacctttcctgagaagccagacttccactgatggaagagctgaaaacacaagcccaggagtgcagacactgccctaattgcttcctgagaattcccatattaataaagaagaaaattatctgccattttttcctactataataatgctgaatcttaatttagagggtacaagggtatggtaatgcttgtagaatctttattatctcaacaatctaaaaaatgatgtttatttccatagtttgatagtgccacttaaatgccaattaaacaagaatataacatttcaatagaaatttttatttcattttcaattactttgtaaattcgtgtgtatttagtacactgatttgtttttttacatttctgctttgaatgcagatgcaatttaatataatagattttttaatgaattaatcttaacatagtaatctttagctttttatacaaatatatttaatttaggagtatatgtgtgtctatacacacacatacataaatataccacatatacacctgatagtcaaataaggtacagaaattttatcttgtcaattatgccaaataatctctttaatgtgcactcaaacatgtaataaactttggataattaaataatgtgccaaatttaagttgtaccacaatattcaaatcatagtcttatggaactctgtaattggacacaaacaaaaacttgtcatataggaatttttttctgtcttttccctgtgtacagcaaactgaggaagaaagatcattatttaatactgggccctgaaatgacacccaatttgttaatggaattatgaagaacagcaatataatacactgaagaataacttagtatcagagaccaataaatcacttttttaaggatgaaccagagagtggatggcagaggtgggctgtagagccaaagtggggtgggagcgcgaagatggcagctgctgaggaggagccgaagcccaaaaagctgaaggtggaggcgccgcaagcgctgagagaaaatattctctttggaatgggaaatcctctgcttgacatctctgctgtagtggacaaagatttccttgataagtattctctgaaaccaaatgaccaaatcttggctgaagacaaacacaaggaactgtttgatgaacttgtgaaaaaattcaaagtcgaatatcatgctggtggctctacccagaattcaattaaagtggctcagtggatgattcaacagccacacaaagcagcaacattttttggatgcattgggatagataaatttggggagatcctgaagagaaaagctgctgaagcccatgtggatgctcattactacgagcagaatgagcagccaacaggaacttgtgctgcatgcatcactggtgacaacaggtccctcatagctaatcttgctgctgccaattgttataaaaaggaaaaacatcttgatctggagaaaaactggatgttggtagaaaaagcaagagtttgttatatagcaggcttttttcttacagtttccccagagtcagtattaaaggtggctcaccatgcttctgaaaacaacaggattttcactttgaatctatctgcaccgtttattagccagttctacaaggaatcattgatgaaagttatgccttatgttgatatactttttggaaatgagacagaagctgccacttttgctagagagcaaggctttgagactaaagacattaaagagatagccaaaaagacacaagccctgccaaagatgaactcaaagaggcagcgaatcgtgatcttcacccaagggagagatgacactataatggctacagaaagtgaagtcactgcttttgctgtcttggatcaagaccagaaagaaattattgataccaatggagctggagatgcatttgttggaggttttctgtctcaactggtctctgacaagcctctgactgaatgtatccgtgctggccactatgcagcaagcatcataattagacggactggctgcacctttcctgagaagccagacttccactgatggaagagctgaaaacacaagcccaggagtgcagacactgccctaattgcttcctgagaattcccatattaataaagaagaaaattatctgccattttttcctactataataatgctgaatcttaatttagagggtacaagggtatggtaatgcttgtagaatctttattatctcaacaatctaaaaaatgatgtttatttccatagtttgatagtgccacttaaatgccaattaaacaagaatataacatttcaatagaaatttttatttcattttcaattactttgtaaattcgtgtgtatttagtacactgatttgtttttttacatttctgctttgaatgcagatgcaatttaatataatagattttttaatgaattaatcttaacatagtaatctttagctttttatacaaatatatttaatttaggagtatatgtgtgtctatacacacacatacataaatataccacatatacacctgatagtcaaataaggtacagaaattttatcttgtcaattatgccaaataatctctttaatgtgcactcaaacatgtaataaactttggataattaaataatgtgccaaatttaagttgtaccacaatattcaaatcatagtcttatggaactctgtaattggacacaaacaaaaacttgtcatataggaatttttttctgtcttttccctgtgtacagcaaactgaggaagaaagatcattatttaatactgggccctgaaatgacacccaatttgttaatggaattatgaagaacagcaatataatacactgaagaataacttagtatcagagaccaataaatcacttttttaaggatga

SEQ ID NO: 2 - DNA, 250 bp, Artificial Sequence
  oligonucleotide
gatgcgaagagggggcgggaccagagagtggatggcagaggtgggctgtagagccaaagtggggtgggagcgcgaagatggcagctgctgaggaggagccgaagcccaaaaagctgaaggtggaggcgccgcaagcgctgaggtgagcgctgccggacttggggaggagggtgacggcgctgcaagcaagccagggcccacgtggggttgcacggccccgacgctgggtggtgtctctcactgccagctt

SEQ ID NO: 3 - DNA, 15 bp, Artificial Sequence
  oligonucleotide
tcacctcagcgcttg

SEQ ID NO: 4 - DNA, 15 bp, Artificial Sequence
  oligonucleotide
tcacctcagcgcttg

SEQ ID NO: 5 - DNA, 15 bp, Artificial Sequence
  oligonucleotide
tcacctcagcgcttg

SEQ ID NO: 6 - DNA, 17 bp, Artificial Sequence
  oligonucleotide
aaagttgcaacatcaat

SEQ ID NO: 7 - DNA, 17 bp, Artificial Sequence
  oligonucleotide
aaagttgcaacatcaat

SEQ ID NO: 8 - DNA, 17 bp, Artificial Sequence
  oligonucleotide
aaagttgcaacatcaat

SEQ ID NO: 9 - DNA, 19 bp, Artificial Sequence
  oligonucleotide
ctttatacttattaggaag

SEQ ID NO: 10 - DNA, 19 bp, Artificial Sequence
  oligonucleotide
ctttatacttattaggaag

SEQ ID NO: 11 - DNA, 19 bp, Artificial Sequence
  oligonucleotide
ctttatacttattaggaag

SEQ ID NO: 12 - DNA, 19 bp, Artificial Sequence
  oligonucleotide
ctttatacttattaggaag

SEQ ID NO: 13 - DNA, 19 bp, Artificial Sequence
  oligonucleotide
ctttatacttattaggaag

SEQ ID NO: 14 - DNA, 19 bp, Artificial Sequence
  oligonucleotide
ctttatacttattaggaag

SEQ ID NO: 15 - DNA, 19 bp, Artificial Sequence
  oligonucleotide
ctttatacttattaggaag

SEQ ID NO: 16 - DNA, 19 bp, Artificial Sequence
  oligonucleotide
ctttatacttattaggaag

SEQ ID NO: 17 - DNA, 19 bp, Artificial Sequence
  oligonucleotide
ctttatacttattaggaag

SEQ ID NO: 18 - DNA, 19 bp, Artificial Sequence
  oligonucleotide
ctttatacttattaggaag

SEQ ID NO: 19 - DNA, 19 bp, Artificial Sequence
  oligonucleotide
ctttatacttattaggaag

SEQ ID NO: 20 - DNA, 18 bp, Artificial Sequence
  oligonucleotide
gttttaaatcagtttgat

SEQ ID NO: 21 - DNA, 18 bp, Artificial Sequence
  oligonucleotide
gttttaaatcagtttgat

SEQ ID NO: 22 - DNA, 18 bp, Artificial Sequence
  oligonucleotide
gttttaaatcagtttgat

SEQ ID NO: 23 - DNA, 18 bp, Artificial Sequence
  oligonucleotide
gttttaaatcagtttgat

SEQ ID NO: 24 - DNA, 18 bp, Artificial Sequence
  oligonucleotide
gttttaaatcagtttgat

SEQ ID NO: 25 - DNA, 18 bp, Artificial Sequence
  oligonucleotide
gttttaaatcagtttgat

SEQ ID NO: 26 - DNA, 18 bp, Artificial Sequence
  oligonucleotide
gttttaaatcagtttgat

SEQ ID NO: 27 - DNA, 18 bp, Artificial Sequence
  oligonucleotide
gttttaaatcagtttgat

SEQ ID NO: 28 - DNA, 18 bp, Artificial Sequence
  oligonucleotide
gttttaaatcagtttgat

SEQ ID NO: 29 - DNA, 18 bp, Artificial Sequence
  oligonucleotide
gttttaaatcagtttgat

SEQ ID NO: 30 - DNA, 18 bp, Artificial Sequence
  oligonucleotide
gttttaaatcagtttgat

SEQ ID NO: 31 - DNA, 17 bp, Artificial Sequence
  oligonucleotide
gctttttaaagcaacag

SEQ ID NO: 32 - DNA, 17 bp, Artificial Sequence
  oligonucleotide
gctttttaaagcaacag

SEQ ID NO: 33 - DNA, 17 bp, Artificial Sequence
  oligonucleotide
gctttttaaagcaacag

SEQ ID NO: 34 - DNA, 17 bp, Artificial Sequence
  oligonucleotide
gctttttaaagcaacag

SEQ ID NO: 35 - DNA, 17 bp, Artificial Sequence
  oligonucleotide
gctttttaaagcaacag

SEQ ID NO: 36 - DNA, 17 bp, Artificial Sequence
  oligonucleotide
gctttttaaagcaacag

SEQ ID NO: 37 - DNA, 17 bp, Artificial Sequence
  oligonucleotide
gctttttaaagcaacag

SEQ ID NO: 38 - DNA, 18 bp, Artificial Sequence
  oligonucleotide
agctttttaaagcaacag

SEQ ID NO: 39 - DNA, 18 bp, Artificial Sequence
  oligonucleotide
agctttttaaagcaacag

SEQ ID NO: 40 - DNA, 18 bp, Artificial Sequence
  oligonucleotide
agctttttaaagcaacag

SEQ ID NO: 41 - DNA, 18 bp, Artificial Sequence
  oligonucleotide
agctttttaaagcaacag

SEQ ID NO: 42 - DNA, 17 bp, Artificial Sequence
  oligonucleotide
ctttgggatttcagaaa

SEQ ID NO: 43 - DNA, 17 bp, Artificial Sequence
  oligonucleotide
ctttgggatttcagaaa

SEQ ID NO: 44 - DNA, 17 bp, Artificial Sequence
  oligonucleotide
ctttgggatttcagaaa

SEQ ID NO: 45 - DNA, 17 bp, Artificial Sequence
  oligonucleotide
ctttgggatttcagaaa

SEQ ID NO: 46 - DNA, 17 bp, Artificial Sequence
  oligonucleotide
ctttgggatttcagaaa

SEQ ID NO: 47 - DNA, 17 bp, Artificial Sequence
  oligonucleotide
ctttgggatttcagaaa

SEQ ID NO: 48 - DNA, 17 bp, Artificial Sequence
  oligonucleotide
ctttgggatttcagaaa

SEQ ID NO: 49 - DNA, 17 bp, Artificial Sequence
  oligonucleotide
ctttgggatttcagaaa

SEQ ID NO: 50 - DNA, 17 bp, Artificial Sequence
  oligonucleotide
ctttgggatttcagaaa

SEQ ID NO: 51 - DNA, 17 bp, Artificial Sequence
  oligonucleotide
caaagctcttttccttg

SEQ ID NO: 52 - DNA, 17 bp, Artificial Sequence
  oligonucleotide
caaagctcttttccttg

SEQ ID NO: 53 - DNA, 17 bp, Artificial Sequence
  oligonucleotide
caaagctcttttccttg

SEQ ID NO: 54 - DNA, 17 bp, Artificial Sequence
  oligonucleotide
caaagctcttttccttg

SEQ ID NO: 55 - DNA, 17 bp, Artificial Sequence
  oligonucleotide
caaagctcttttccttg

SEQ ID NO: 56 - DNA, 17 bp, Artificial Sequence
  oligonucleotide
caaagctcttttccttg

SEQ ID NO: 57 - DNA, 17 bp, Artificial Sequence
  oligonucleotide
aacttcagaaacactta

SEQ ID NO: 58 - DNA, 17 bp, Artificial Sequence
  oligonucleotide
aacttcagaaacactta

SEQ ID NO: 59 - DNA, 18 bp, Artificial Sequence
  oligonucleotide
gaacttcagaaacactta

SEQ ID NO: 60 - DNA, 18 bp, Artificial Sequence
  oligonucleotide
gaacttcagaaacactta

SEQ ID NO: 61 - DNA, 18 bp, Artificial Sequence
  oligonucleotide
gaacttcagaaacactta

SEQ ID NO: 62 - DNA, 18 bp, Artificial Sequence
  oligonucleotide
gaacttcagaaacactta

SEQ ID NO: 63 - DNA, 18 bp, Artificial Sequence
  oligonucleotide
gaacttcagaaacactta

SEQ ID NO: 64 - DNA, 18 bp, Artificial Sequence
  oligonucleotide
gaacttcagaaacactta

SEQ ID NO: 65 - DNA, 18 bp, Artificial Sequence
  oligonucleotide
gaacttcagaaacactta

SEQ ID NO: 66 - DNA, 18 bp, Artificial Sequence
  oligonucleotide
gaacttcagaaacactta

SEQ ID NO: 67 - DNA, 18 bp, Artificial Sequence
  oligonucleotide
gaacttcagaaacactta

SEQ ID NO: 68 - DNA, 14 bp, Artificial Sequence
  oligonucleotide
aaaatggccgcgcc

SEQ ID NO: 69 - DNA, 14 bp, Artificial Sequence
  oligonucleotide
aaaatggccgcgcc

SEQ ID NO: 70 - DNA, 14 bp, Artificial Sequence
  oligonucleotide
aaaatggccgcgcc

SEQ ID NO: 71 - DNA, 14 bp, Artificial Sequence
  oligonucleotide
aaaatggccgcgcc

SEQ ID NO: 72 - DNA, 14 bp, Artificial Sequence
  oligonucleotide
aaaatggccgcgcc

SEQ ID NO: 73 - DNA, 14 bp, Artificial Sequence
  oligonucleotide
aaaatggccgcgcc

SEQ ID NO: 74 - DNA, 14 bp, Artificial Sequence
  oligonucleotide
cagctgccatcttc

SEQ ID NO: 75 - DNA, 14 bp, Artificial Sequence
  oligonucleotide
cagctgccatcttc

SEQ ID NO: 76 - DNA, 14 bp, Artificial Sequence
  oligonucleotide
cagctgccatcttc

SEQ ID NO: 77 - DNA, 15 bp, Artificial Sequence
  oligonucleotide
gcagctgccatcttc

SEQ ID NO: 78 - DNA, 15 bp, Artificial Sequence
  oligonucleotide
gcagctgccatcttc

SEQ ID NO: 79 - DNA, 15 bp, Artificial Sequence
  oligonucleotide
gcagctgccatcttc

SEQ ID NO: 80 - DNA, 14 bp, Artificial Sequence
  oligonucleotide
gcagctgccatctt

SEQ ID NO: 81 - DNA, 14 bp, Artificial Sequence
  oligonucleotide
gcagctgccatctt

SEQ ID NO: 82 - DNA, 14 bp, Artificial Sequence
  oligonucleotide
gcagctgccatctt

SEQ ID NO: 83 - DNA, 14 bp, Artificial Sequence
  oligonucleotide
cacctcagcgcttg

SEQ ID NO: 84 - DNA, 14 bp, Artificial Sequence
  oligonucleotide
cacctcagcgcttg

SEQ ID NO: 85 - DNA, 14 bp, Artificial Sequence
  oligonucleotide
cacctcagcgcttg

SEQ ID NO: 86 - DNA, 15 bp, Artificial Sequence
  oligonucleotide
tcacctcagcgcttg

SEQ ID NO: 87 - DNA, 15 bp, Artificial Sequence
  oligonucleotide
tcacctcagcgcttg

SEQ ID NO: 88 - DNA, 15 bp, Artificial Sequence
  oligonucleotide
tcacctcagcgcttg

SEQ ID NO: 89 - DNA, 16 bp, Artificial Sequence
  oligonucleotide
ctcacctcagcgcttg

SEQ ID NO: 90 - DNA, 16 bp, Artificial Sequence
  oligonucleotide
ctcacctcagcgcttg

SEQ ID NO: 91 - DNA, 16 bp, Artificial Sequence
  oligonucleotide
ctcacctcagcgcttg

SEQ ID NO: 92 - DNA, 14 bp, Artificial Sequence
  oligonucleotide
tcacctcagcgctt

SEQ ID NO: 93 - DNA, 14 bp, Artificial Sequence
  oligonucleotide
tcacctcagcgctt

SEQ ID NO: 94 - DNA, 14 bp, Artificial Sequence
  oligonucleotide
tcacctcagcgctt

SEQ ID NO: 95 - DNA, 15 bp, Artificial Sequence
  oligonucleotide
ctcacctcagcgctt

SEQ ID NO: 96 - DNA, 15 bp, Artificial Sequence
  oligonucleotide
ctcacctcagcgctt

SEQ ID NO: 97 - DNA, 15 bp, Artificial Sequence
  oligonucleotide
ctcacctcagcgctt

SEQ ID NO: 98 - DNA, 14 bp, Artificial Sequence
  oligonucleotide
ctcacctcagcgct

SEQ ID NO: 99 - DNA, 14 bp, Artificial Sequence
  oligonucleotide
ctcacctcagcgct

SEQ ID NO: 100 - DNA, 14 bp, Artificial Sequence
  oligonucleotide
ctcacctcagcgct

SEQ ID NO: 101 - DNA, 15 bp, Artificial Sequence
  oligonucleotide
tcacctcagcgcttg

SEQ ID NO: 102 - DNA, 15 bp, Artificial Sequence
  oligonucleotide
tcacctcagcgcttg

SEQ ID NO: 103 - DNA, 17 bp, Artificial Sequence
  oligonucleotide
aaagttgcaacatcaat

SEQ ID NO: 104 - DNA, 18 bp, Artificial Sequence
  oligonucleotide
gttttaaatcagtttgat

SEQ ID NO: 105 - DNA, 17 bp, Artificial Sequence
  oligonucleotide
gctttttaaagcaacag

SEQ ID NO: 106 - DNA, 18 bp, Artificial Sequence
  oligonucleotide
agctttttaaagcaacag

SEQ ID NO: 107 - DNA, 17 bp, Artificial Sequence
  oligonucleotide
ctttgggatttcagaaa

SEQ ID NO: 108 - DNA, 17 bp, Artificial Sequence
  oligonucleotide
caaagctcttttccttg

SEQ ID NO: 109 - DNA, 17 bp, Artificial Sequence
  oligonucleotide
aacttcagaaacactta

SEQ ID NO: 110 - DNA, 18 bp, Artificial Sequence
  oligonucleotide
gaacttcagaaacactta

SEQ ID NO: 111 - DNA, 14 bp, Artificial Sequence
  oligonucleotide
aaaatggccgcgcc

SEQ ID NO: 112 - DNA, 14 bp, Artificial Sequence
  oligonucleotide
cagctgccatcttc

SEQ ID NO: 113 - DNA, 15 bp, Artificial Sequence
  oligonucleotide
gcagctgccatcttc

SEQ ID NO: 114 - DNA, 14 bp, Artificial Sequence
  oligonucleotide
gcagctgccatctt

SEQ ID NO: 115 - DNA, 14 bp, Artificial Sequence
  oligonucleotide
cacctcagcgcttg

SEQ ID NO: 116 - DNA, 16 bp, Artificial Sequence
  oligonucleotide
ctcacctcagcgcttg

SEQ ID NO: 117 - DNA, 14 bp, Artificial Sequence
  oligonucleotide
tcacctcagcgctt

SEQ ID NO: 118 - DNA, 15 bp, Artificial Sequence
  oligonucleotide
ctcacctcagcgctt

SEQ ID NO: 119 - DNA, 14 bp, Artificial Sequence
  oligonucleotide
ctcacctcagcgct

SEQ ID NO: 120 - DNA, 250 bp, Artificial Sequence; identical to SEQ ID NO: 2
  oligonucleotide

SEQ ID NO: 121 - DNA, 74 bp, Artificial Sequence
  oligonucleotide
gaagatggcagctgctgaggaggagccgaagcccaaaaagctgaaggtggaggcgccgcaagcgctgaggtgag

SEQ ID NO: 122 - DNA, 15 bp, Artificial Sequence
  oligonucleotide
caagcgctgaggtga

SEQ ID NO: 123 - DNA, 17 bp, Artificial Sequence
  oligonucleotide
attgatgttgcaacttt

SEQ ID NO: 124 - DNA, 19 bp, Artificial Sequence
  oligonucleotide
cttcctaataagtataaag

SEQ ID NO: 125 - DNA, 18 bp, Artificial Sequence
  oligonucleotide
atcaaactgatttaaaac

SEQ ID NO: 126 - DNA, 17 bp, Artificial Sequence
  oligonucleotide
ctgttgctttaaaaagc

SEQ ID NO: 127 - DNA, 18 bp, Artificial Sequence
  oligonucleotide
ctgttgctttaaaaagct

SEQ ID NO: 128 - DNA, 17 bp, Artificial Sequence
  oligonucleotide
tttctgaaatcccaaag

SEQ ID NO: 129 - DNA, 17 bp, Artificial Sequence
  oligonucleotide
caaggaaaagagctttg

SEQ ID NO: 130 - DNA, 17 bp, Artificial Sequence
  oligonucleotide
taagtgtttctgaagtt

SEQ ID NO: 131 - DNA, 18 bp, Artificial Sequence
  oligonucleotide
taagtgtttctgaagttc

SEQ ID NO: 132 - DNA, 14 bp, Artificial Sequence
  oligonucleotide
ggcgcggccatttt

SEQ ID NO: 133 - DNA, 18 bp, Artificial Sequence
  oligonucleotide
agctttttaaagcaacag

SEQ ID NO: 134 - DNA, 18 bp, Artificial Sequence
  oligonucleotide
agctttttaaagcaacag

SEQ ID NO: 135 - DNA, 18 bp, Artificial Sequence
  oligonucleotide
agctttttaaagcaacag

SEQ ID NO: 136 - DNA, 19 bp, Artificial Sequence
  oligonucleotide
ctttatacttattaggaag

SEQ ID NO: 137 - DNA, 19 bp, Artificial Sequence
  oligonucleotide
ctttatacttattaggaag

SEQ ID NO: 138 - DNA, 19 bp, Artificial Sequence
  oligonucleotide
ctttatacttattaggaag

SEQ ID NO: 139 - DNA, 17 bp, Artificial Sequence
  oligonucleotide
ctttgggatttcagaaa

SEQ ID NO: 140 - DNA, 17 bp, Artificial Sequence
  oligonucleotide
ctttgggatttcagaaa

SEQ ID NO: 141 - DNA, 17 bp, Artificial Sequence
  oligonucleotide
ctttgggatttcagaaa

SEQ ID NO: 142 - DNA, 18 bp, Artificial Sequence
  oligonucleotide
gaacttcagaaacactta

SEQ ID NO: 143 - DNA, 18 bp, Artificial Sequence
  oligonucleotide
gaacttcagaaacactta

SEQ ID NO: 144 - DNA, 17 bp, Artificial Sequence
  oligonucleotide
gctttttaaagcaacag

SEQ ID NO: 145 - DNA, 17 bp, Artificial Sequence
  oligonucleotide
gctttttaaagcaacag

SEQ ID NO: 146 - DNA, 18 bp, Artificial Sequence
  oligonucleotide
gttttaaatcagtttgat

SEQ ID NO: 147 - DNA, 18 bp, Artificial Sequence
  oligonucleotide
gttttaaatcagtttgat

(19) United States
(12) Patent Application Publication
(10) Pub. No.: US 2024/0240188 A1
(43) Pub. Date: Jul. 18, 2024

(54) ANTISENSE OLIGONUCLEOTIDES TARGETING ADENOSINE KINASE

(71) Applicant: Neumirna Therapeutics ApS, København SV (DK)

(72) Inventors: Markus Sakari Kauppinen, Holte (DK); Lykke Pedersen, København NV (DK); Stine Normann Hansen, København S (DK); Henrik Valdemar Klitgaard, Hellerup (DK)

(21) Appl. No.: 18/565,432

(22) PCT Filed: Jun. 3, 2022
(86) PCT No.: PCT/EP2022/065217

§ 371 Date: Nov. 29, 2023

(30) Foreign Application Priority Data
Jun. 4, 2021 (DK) PA202170290

(51) Int. Cl. C12N 15/113; A61K 45/06; A61P 25/00; A61P 25/08

(52) U.S. Cl. CPC C12N 15/1137; A61K 45/06; A61P 25/00; A61P 25/08; C12N 2310/11; C12N 2310/315; C12N 2310/321; C12N 2310/322

(57) ABSTRACT

The present invention provides antisense oligonucleotides targeting adenosine kinase. Such antisense oligonucleotides are useful in treatment of a range of neurological diseases, such as epilepsy. Compositions and methods of treating neurological diseases using the oligonucleotides of the invention are provided.

### FIELD OF THE INVENTION

The present invention provides novel antisense oligonucleotides targeting adenosine kinase. The compounds are useful for treatment of neurological diseases such as epilepsy.

### BACKGROUND

Epilepsy is a serious, chronic neurologic disorder characterised by recurrent spontaneous seizures affecting about 50 million people worldwide.

Present available anti-epileptic drugs, typically control seizures in two-thirds of patients, but probably have no effect on the underlying pathophysiology. The remaining one-third of patients with epilepsy are either drug-resistant or suffer from serious side effects from the presently available drugs.

Brain surgery, vagus nerve stimulation, intracranial stimulation and ketogenic diet represents alternatives to avoid seizures in patients without the option of getting drug treatment.

The development of symptomatic (acquired) epilepsy is thought to involve altered expression of ion channels and neurotransmitter receptors, synaptic remodelling, inflammation, gliosis and neuronal death, among others. However, few anti-epileptogenic interventions targeting these processes have shown sufficient efficacy in vivo, and our understanding of the cell and molecular mechanisms remains incomplete. There are currently no prophylactic treatments (“anti-epileptogenic”) following a brain injury likely to cause development of epilepsy. Similarly, there are no specific neuroprotective treatments for status epilepticus (SE), or treating acute neurologic injuries likely to cause brain damage or epilepsy, for example, stroke, or trauma.

There is therefore a high unmet need for treatments or preventative measures that specifically target the process by which epilepsy and other neurological injuries likely to cause brain damage develop and that overcome some of the above-mentioned problems.

### Adenosine and Adenosine Kinase

Adenosine is a well-characterized endogenous anticonvulsant and seizure terminator of the brain. Adenosine affects seizure generation (ictogenesis), development of epilepsy and its progression (epileptogenesis). Maladaptive changes in adenosine metabolism, in particular increased expression of the astroglial enzyme adenosine kinase (ADK), play a major role in epileptogenesis. (Weltha et al, 2019, The role of adenosine in epilepsy, Brain Res Bull 2019 September, page 1-22.)

Two ADK isoforms, which differ at the N-terminal ends are expressed in mammalian cells. ADK plays a central role in regulating the intracellular and interstitial concentrations of the purine nucleoside adenosine, which exhibits potent cardioprotective and neuroprotective activity. The expression of adenosine kinase undergoes rapid coordinated changes in the brain following epileptic seizures or stroke, resulting in an acute surge of adenosine, which serves to minimize damage to the brain. The two isoforms of adenosine kinase in mammalian cells differ from each other only in their N-termini. The long isoform (AdK-L) contains an extra 20-21 amino acids instead of the first four amino acids of the AdK-short (AdK-S) isoform. The N-terminal extension in the AdK-L functions as a nuclear localization signal. Thus, of the two isoforms, AdK-L is targeted to the nucleus, whereas AdK-S is localised in the cytoplasm. (Cui et al, 2011, Molecular Characterization of Chinese Hamster Cells Mutants Affected in Adenosine Kinase and Showing Novel Genetic and Biochemical Characteristics, BMC Biochemistry 2011.)

Adenosine exerts a variety of cardioprotective effects. Further, dysregulation of ADK expression and resulting disruption of adenosine homeostasis is implicated in a wide range of neurologic and neuropsychiatric pathologies. Astroglial ADK is a promising target for the prediction and prevention of seizures in epilepsy. Astrogliosis and associated overexpression of ADK have also been identified in a rat model of severe traumatic brain injury (TBI) induced by a lateral fluid percussion injury. Further, ADK expression levels critically determine the brain's vulnerability to the effects of a stroke. Sleep and the intensity of sleep are also enhanced by adenosine and its receptor agonists, whereas antagonists such as caffeine or theophylline induce wakefulness.

According to Boison et al., the link between overexpression of ADK and cognitive impairment might be of pathologic relevance for neurologic conditions in which overexpression of ADK has either been confirmed (epilepsy) or suspected (Alzheimer's disease, Parkinson's disease, amyotrophic lateral sclerosis). The adenosine hypothesis of schizophrenia postulates that hypofunction of adenosine signaling may contribute to the pathophysiology of schizophrenia. In diabetes mellitus, adenosine homeostasis is critically altered in several tissues.

Further, homeostasis of adenosine receptor signaling is of crucial importance in the regulation of inflammation and the release of proinflammatory cytokines. The homoeostasis of adenosine receptor signaling is also of critical significance for the chronic inflammatory reactions in IBD.

The role of the adenosine/ADK regulatory system in cancer may depend on the type of cancer. ADK activity was found to be reduced in hepatoma cells, suggesting that increased adenosine might provide a selective advantage for hepatic cancers. (Boison et al., 2013, Adenosine Kinase: Exploitation for Therapeutic Gain, Pharmacol Rev 65:906-943, July 2013.)

### Adenosine Receptors

Activation of inhibitory adenosine A1 receptors is beneficial in epilepsy, chronic pain and cerebral ischemia, and inhibition of facilitatory A2A receptors has profound neuroprotective effects. (Boison et al, 2008, Adenosine as a neuromodulator in neurological diseases, Curr Opin Pharmacol, 2008 February.)

Adenosine is a neuromodulator that operates via the most abundant inhibitory adenosine A1 receptors (A1Rs) and the less abundant, but widespread, facilitatory A2ARs. It is commonly assumed that A1Rs play a key role in neuroprotection since they decrease glutamate release and hyperpolarize neurons. (Rodrigo A. Cunha, 2005, Neuroprotection by adenosine in the brain: From A1 receptor activation to A2A receptor blockade, Purinergic Signalling (2005) 1: 111-134.)

Restoring A3AR signaling in the spinal cord by inhibiting adenosine kinase or activating A3AR with intrathecal selective A3AR agonists prevent the establishment chemotherapy-induced neuropathic pain (CINP). (Wahlman et al, 2018, Chemotherapy-induced pain is promoted by enhanced spinal adenosine kinase levels via astrocyte-dependent mechanisms, Pain. 2018 June; 159(6): 1025-1034.)

### Epilepsy, Neuroprotection and Psychiatric Disorders

Adenosine has an anticonvulsant and neuroprotective effect. (Patodia et al, 2020, Adenosine kinase and adenosine receptors A1R and A2AR in temporal lobe epilepsy and hippocampal sclerosis and association with risk factors for SUDEP, Epilepsia, page 787-797.)

Focal adenosine augumentation therapy, using adenosine kinase inhibitor, has been proved to be effective for reducing seizures in both animal models and in human brain tissue resected from a variety of etiology of refractory epilepsy patients. In addition to reducing seizures, adenosine augumentation therapy can also palliate co-morbidities, like sleep, cognition, or depression. Transgenic mice with reduced ADK were resistant to epileptogenesis induced by acute brain injury. (Wang et al, 2020, Role of Adenosine Kinase Inhibitor in Adenosine Augmentation Therapy for Epilepsy: A Potential Novel Drug for Epilepsy, Current Drug Targets, abstract.)

According to Boison et al. 2006, adenosine is an inhibitory modulator of brain activity with neuroprotective and anticonvulsant properties. Thus, cell-based delivery of adenosine holds great promise for novel therapies for epilepsy and stroke. (Boison et al, 2013, Adenosine kinase, epilepsy and stroke: mechanisms and therapies, Trends Pharmacol Sci, Abstract.) Adenosine kinase also has a developmental role in mediating behaviors in adulthood related to neuropsychiatric disease. (Osborne et al, 2018, Developmental role of adenosine kinase for the expression of sex-dependent neuropsychiatric behaviour, Neuropharmacology, 2018 October.) [schizophrenia, autism, ADHD]

A study by Hai-Ying Shen et al 2012 found that augmentation of adenosine by pharmacologic inhibition of adenosine kinase exerted antipsychotic-like activity in mice. Furthermore, overexpression of ADK in transgenic mice was associated with attentional impairments linked to schizophrenia. (Hai-Ying Shen et al 2012, Adenosine augmentation ameliorates psychotic and cognitive endophenotypes of schizophrenia, J Clin Invest, page 2567-2577.)

### Pain

According to Otsuguro et al. 2015, an adenosine kinase inhibitor is a potential candidate for controlling pain. (Otsuguro et al., 2015, An adenosine kinase inhibitor, ABT-702, inhibits spinal nociceptive transmission by adenosine release via equilibrative nucleoside transporters in rat, neuropharmacology volume 97, abstract.) Inhibitors of adenosine kinase enhance extracellular concentrations of the inhibitory neuromodulator adenosine at sites of tissue hyperexcitability and produce antinociceptive effects in animal models of pain and inflammation. Furthermore, adenosine kinase inhibitors produce specific antihyperalgesic effects. (Jarvis et al, 2002, Comparison of the ability of adenosine kinase inhibitors and adenosine receptor agonists to attenuate thermal hyperalgesia and reduce motor performance in rats, Pharmacology Biochemistry and Behavior vol 73, abstract.)

Adenosine kinase inhibitors have shown antinociceptive activity in a variety of animal models of nociception and novel adenosine kinase inhibitor A-134974 potently reduces tactile allodynia. (Zhu et al, 2001, A-134974: a novel adenosine kinase inhibitor, relieves tactile allodynia via spinal sites of action in peripheral nerve injured rats, Brain Research vol 905, abstract.) Adenosine kinase inhibitors have also been shown to provide effective antinociceptive, antiinflammatory and anticonvulsant activity in animal models, thus suggesting their potential therapeutic utility for pain, inflammation, epilepsy and possibly other central and peripheral nervous system diseases associated with cellular trauma and inflammation. (Gomtsyan et al, 2004, Non-nucleoside inhibitors of adenosine kinase, Current Pharmaceutical Design, abstract.)

According to Bauser et al. 2004, adenosine kinase inhibition is an attractive therapeutic approach for several conditions for example, neurodegeneration, seizures, ischemia, inflammation and pain. (Bauser er al, 2004, Discovery and optimization of 2-aryl oxazolo-pyrimidines as adenosine kinase inhibitors using liquid phase parallel synthesis, Bioorganic & Medicinal Chemistry Letters, abstract.)

### Encephalitis

Rasmussen encephalitis is a rare neurological disorder characterized by unilateral inflammation of cerebral cortex and other structures, most notably the hippocampus, progressive cognitive deterioration, and pharmacoresistant focal epilepsy. Luan et al. suggest that overexpression of adenosine kinase is a common pathologic hallmark of Rasmussen encephalitis, and that upregulation of neuronal A1R in Rasmussen encephalitis is crucial in preventing the spread of seizures. Furthermore, adenosine acts as an endogenous neuromodulator with anticonvulsion, antiinflammation, and restoring cognitive function when cognition is impaired secondary to epilepsy. Disruption of adenosine homeostasis has been linked with epilepsy, inflammation and cognitive dysfunction. It has been proved that the alteration of adenosine receptors and the major adenosine-removing enzyme ADK contribute to the disruption of adenosine homeostasis in epilepsy. (Luan et al, 2017, Upregulation of Neuronal Adenosine A1 Receptor in Human Rasmussen Encephalitis, J Neuropathol Exp Neurol vol 76, page 720-731.)

### Angiogenesis

Targeting adenosine kinase to elevate intracellular adenosine promotes endothelial proliferation and migration in vitro as well as vessel sprouting ex vivo. Additionally, endothelial specific adenosine kinase knockout mice have increased retinal angiogenesis, accelerated wound healing, and were protected against hindlimb ischemic injury. (Xu et al., 2017, Intracellular adenosine regulates epigenetic programming in endothelial cells to promote angiogenesis, EMBO Molecular Medicine, page 1263-1278.)

### Cancer

A study by Huang et al 2015 suggested that adenosine kinase is involved in glioma progression, and that increased adenosine kinase levels in peritumoral tissues may be associated with epilepsy in glioma. (Huang et al, 2015, Adenosine deaminase and adenosine kinase expression in human glioma and their correlation with glioma-associated epilepsy, Molecular Medicine Reports 12, page 6509-6516.)

### Diabetes, Inflammation, Cardiovascular Disorders, Kidney Disorders and Lung Disorders

According to Pye et al 2014, adenosine provides anti-inflammatory effects in cardiovascular disease via activation of adenosine A2A receptors; however, the physiological effect of adenosine could be limited due to its phosphorylation by adenosine kinase. Treatment with the adenosine kinase inhibitor ABY702 reduced blood glucose level in diabetic mice, reduced albuminuria and markers of glomerular injury, nephrinuria and podocalyxin excretion levels, in diabetic mice. Further, indices of oxidative stress were reduced. (Pye et al, 2014, Adenosine Kinase Inhibition Protects The Kidney Against Streptozotocin-Induced Diabetes Through Anti-inflammatory and Anti-oxidant Mechanisms, Pharmacol Res.)

Activation of A1 adenosine receptor protects against acute kidney injury by improving renal hemodynamic alterations, decreasing tubular necrosis and its inhibition might facilitate removal of toxin or drug metabolite in chronic kidney disease mode. (Pandey et al, 2021, “Adenosine an old player with new possibilities in kidney diseases”: Preclinical evidences and clinical perspectives, Life Sciences vol 265, abstract.)

In many therapeutic areas modulation of adenosine function has been viewed as a therapeutic option, e.g., neuropathic pain, stroke, asthma, chronic obstructive pulmonary disease (COPD) and sleep promotion. (Knutsen et al, 2007, Therapeutic Areas I: Central Nervous System, Pain, Metabolic Syndrome, Urology, Gastrointestinal and Cardiovascular, Comprehensive Medicinal Chemistry II, 2007, https://www.sciencedirect.com/topics/medicine-and-dentistry/adenosine-kinase-inhibitor, accessed 21 Apr. 2021.)

### SUMMARY OF THE INVENTION

There is a high unmet medical need for improved treatments of neurological diseases, as many of the diseases can not be treated in a sufficient manner, or where presently available treatments cause serious side effects. The compounds of the invention, are potent inhibitors of Adenosine Kinase (ADK), such as ADK-L and thereby useful for treatment of neurological diseases such as epilepsy.

### FIGURE LEGENDS

FIG. 1. qPCR primers and probes.

FIG. 2.1 Ranking of the A) ADK-L gapmer and B) mixmer antisense oligonucleotides from the highest to lowest level of ADK-L knockdown. The horizontal dotted line shows 100% (no knockdown) of RNAiMAX only treated cells. A) The black line represents 70% knockdown and the grey line 80% knockdown. The vertical dotted line shows the cut off for oligonucleotides selected for confirmation. n, N=1, 1-2, mean±SEM

FIG. 2.2 Ranking of the selected ADK-L gapmer antisense oligonucleotides from the highest to lowest level of knockdown in A) Primary qPCR assay and B) Secondary qPCR assay. The horizontal dotted line shows 100% (no knockdown) of only RNAiMAX treated cells and the grey line shows 80% knockdown. n, N=2,3-4, mean±SEM

FIG. 3.1 Dose-response study. The horizontal dotted line shows 100% (no knockdown) of RNAiMAX only treated cells and the black line shows 80% knockdown. n, N=1-2,2-4, mean±SEM

FIG. 4.1 Dose-response curves and IC50 values, 3-parameter non-linear curve fit, n, N=2-3,4-6; all technical replicates are depicted. The horizontal dotted line represents 50% knockdown.

FIG. 5.1. Normalized mRNA expression values for ADK (both isoforms), n=3.

FIG. 5.2. Differential gene expression analysis of cells treated with Seq ID 42 (left) and Seq ID 139 (right). The volcano plots show the different levels of transcripts between Seq ID 42, Seq ID 139 and mock treated cells, correlating the changes in RNA expression between antisense oligonucleotide-treated and mock-treated groups with the significance of the differential expression. The x-axis denoted relative change in expression while the y-axis denotes the significance. Each dot denotes a specific RNA. Black dots represent non-significant changes, while grey dots display significant values.

FIG. 5.3. In silico analysis using the antisense oligonucleotide sequences to predict all potential target sites within the spliced transcriptome (cytoplasmic; column 1-4) and the unspliced transcriptome (nuclear, column 5-8). This was done for either 1) perfect match in target mRNAs to the antisense oligonucleotide, or 2) sequences with 1, 2, 3 or 4 mismatches (INDELs). The resulting predicted targets were compared to the results from the RNAseq data (table 2, 3 nM and 3, 20 nM), to see how many of the predicted target mRNAs (row 1) were expressed in the data set (row 2) and of these significantly how many were differentially expressed (row 3), and whether the DGE constitute upregulation (row 4 and 5) or downregulation (row 6 and 7) in the data set.

FIG. 5.4. Close-up of ADK-L to ADK-S first exons. IGV genome browser views showing read mapping on first exons of ADK-L (A) and ADK-S (B). Dotted horizontal lines differentiate between different treatment groups with n=3.

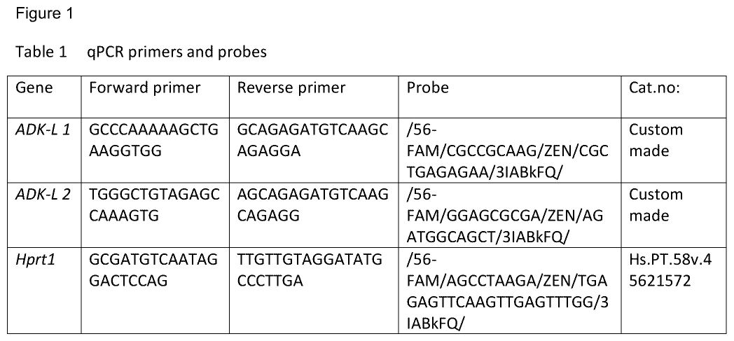

### DETAILED DESCRIPTION OF THE INVENTION

In describing the embodiments of the invention, specific terminology will be resorted to for the sake of clarity. However, the invention is not intended to be limited to the specific terms so selected, and it is understood that each specific term includes all technical equivalents, which operate in a similar manner to accomplish a similar purpose.

The term “therapeutically effective amount”, or “effective amount” or effective dose”, refers to an amount of a therapeutic agent, which confers a desired therapeutic effect on an individual in need of the agent. The effective amount may vary among individuals depending on the health and physical condition of the individual to be treated, the taxonomic group of the individuals to be treated, the formulation of the composition, the method of administration, assessment of the individual's medical condition, and other relevant factors.

The term “treatment” refers to any administration of a therapeutic medicament, herein comprising an antisense oligonucleotide that partially or completely cures or reduces one or more symptoms or features of a given disease.

The term “compound” as used herein, refers to a compound comprising an oligonucleotide according to the invention. In some embodiments, a compound may comprise other elements a part from the oligonucleotide of the invention. Such other elements may in non-limiting example be a delivery vehicle which is conjugated or in other way bound to the oligonucleotide.

“Antisense oligonucleotide” means a single-stranded oligonucleotide having a nucleobase sequence that permits hybridization to a corresponding region or segment of a target nucleic acid. In some instances, the antisense oligonucleotide of the present invention is a “mixmer”, and in some instances, the antisense oligonucleotide of the present invention is a “gapmer”.

A “mixmer” is an antisense oligonucleotide, comprising a mix of nucleoside analogues such as LNA and DNA nucleosides, and wherein the antisense oligonucleotide does not comprise an internal region having a plurality of nucleosides such as a contiguous stretch of not more than 4 or 5 DNA nucleotides. A mixmer is not capable of recruiting an RNAse, such as RNAseH, but rather exerts its effect by binding to the target RNA and thereby blocking its normal function.

A “gapmer” is an antisense oligonucleotide, comprising a contiguous stretch of at least 6 or 7 DNA nucleotides of nucleoside flanked by stretches of nucleotides comprising affinity enhancing nucleotide analogues such as LNA nucleosides. A gapmer is capable of recruiting an RNAse, such as RNAseH, wherein the nucleosides comprising the internal region are chemically distinct from the nucleoside or nucleosides comprising the external wings.

“Nucleoside analogues” are described by e.g. Freier & Altmann; Nucl. Acid. Res., 1997, 25, 4429-4443 and Uhlmann; Curr. Opinion in Drug Development, 2000, 3(2), 293-213, and examples of suitable and preferred nucleoside analogues are provided by WO2007031091, which are hereby incorporated by reference.

“5-methylcytosine” means a cytosine modified with a methyl group attached to the 5′ position. A 5-methylcytosine is a modified nucleobase often replacing cytosine in antisense oligonucleotides. It is within the scope of the present invention that in the oligonucleotides of the invention, cytosine is replaced with 5-methylcytosine.

“2′-O-methoxyethyl” (also 2′-MOE and 2′-O(CH˜)˜-OCH3) refers to an O-methoxy-ethyl modification at the 2′ position of a furanose ring.

“2′-MOE nucleoside” (also 2′-O-methoxyethyl nucleoside) means a nucleoside comprising a 2′-MOE modified sugar moiety.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides. In some aspects bicyclic nucleoside analogues are LNA nucleotides, and these terms may therefore be used interchangeably, and in such embodiments, both are characterized by the presence of a linker group (such as a bridge) between C2′ and C4′ of the ribose sugar ring. When used in the present context, the terms “LNA unit”, “LNA monomer”, “LNA residue”, “locked nucleic acid unit”, “locked nucleic acid monomer” or “locked nucleic acid residue”, refer to a bicyclic nucleoside analogue. LNA units are described in inter alia WO 99/14226, WO 00/56746, WO 00/56748, WO 01/25248, WO 02/28875, WO 03/006475, WO2015071388, and WO 03/095467.

“Beta-D-Oxy LNA”, is a preferred LNA variant.

“Bicyclic nucleic acid” or “BNA” or “BNA nucleosides” mean nucleic acid monomers having a bridge connecting two carbon atoms between the 4′ and 2′ position of the nucleoside sugar unit, thereby forming a bicyclic sugar. Examples of such bicyclic sugar include, but are not limited to A) pt-L-methyleneoxy (4′-CH2-O-2′) LNA, (B) P-D-Methyleneoxy (4′-CH2-O-2′) LNA, (C) Ethyleneoxy (4′-(CH2)2-O-2′) LNA, (D) Aminooxy (4′-CH2-O—N(R)-2′) LNA and (E) Oxyamino (4′-CH2-N(R)—O-2′) LNA.

As used herein, LNA compounds include, but are not limited to, compounds having at least one bridge between the 4′ and the 2′ position of the sugar wherein each of the bridges independently comprises 1 or from 2 to 4 linked groups independently selected from —[C(R˜)(R2)]ₙ-, —C(R˜)═C(R2)-, —C(R˜)=N, —C(═NREM)-, —C(═O)—, —C(═S)—, —O—, —Si(Ri)q-, —S(═O)— and —N(R&)-; wherein: x is 0, 1, or 2; n is 1, 2, 3, or 4; each R& and R2 is, independently, H, a protecting group, hydroxyl, C>>C>> alkyl, substituted C>> (—CHz-) group connecting the 2′ oxygen atom and the 4′ carbon atom, for which the term methyleneoxy (4′-CH&-O-2′) LNA is used.

Furthermore; in the case of the bicyclic sugar moiety having an ethylene bridging group in this position, the ethyleneoxy (4′-CH&CH&-O-2′) LNA is used. n-L-methyleneoxy (4′-CH&-O-2′), an isomer of methyleneoxy (4′-CH&-O-2′) LNA is also encompassed within the definition of LNA, as used herein.

In some embodiments, the nucleoside unit is an LNA unit selected from the list of beta-D-oxy-LNA, alpha-L-oxy-LNA, beta-D-amino-LNA, alpha-L-amino-LNA, beta-D-thio-LNA, alpha-L-thio-LNA, 5′-methyl-LNA, beta-D-ENA and alpha-L-ENA.

“cEt” or “constrained ethyl” means a bicyclic sugar moiety comprising a bridge connecting the 4′-carbon and the 2′-carbon, wherein the bridge has the formula: 4′-CH(CHq)-O-2′.

“Constrained ethyl nucleoside” (also cEt nucleoside) means a nucleoside comprising a bicyclic sugar moiety comprising a 4′-CH(CH3)-O-2′ bridge. cEt and some of its properties are described in Pallan et al. Chem Commun (Camb). 2012, August 25; 48(66): 8195-8197.

“Tricyclo (tc)-DNA” belongs to the class of conformationally constrained DNA analogs that show enhanced binding properties to DNA and RNA. Structure and method of production may be seen in Renneberg et al. Nucleic Acids Res. 2002 Jul. 1; 30(13): 2751-2757.

“2′-fluoro”, as referred to herein is a nucleoside comprising a fluoro group at the 2′ position of the sugar ring. 2′-fluorinated nucleotides are described in Peng et al. J Fluor Chem. 2008 September; 129(9): 743-766.

“2′-O-methyl”, as referred to herein, is a nucleoside comprising a sugar comprising an —OCH3 group at the 2′ position of the sugar ring.

“Conformationally Restricted Nucleosides (CRN)” and methods for their synthesis, as referred to herein, are described in WO2013036868, which is hereby incorporated by reference. CRN are sugar-modified nucleosides, in which, similar to LNA, a chemical bridge connects the C2′ and C4′ carbons of the ribose. However, in a CRN, the C2′-C4′ bridge is one carbon longer than in an LNA molecule. The chemical bridge in the ribose of a CRN locks the ribose in a fixed position, which in turn restricts the flexibility of the nucleobase and phosphate group. CRN substitution within an RNA- or DNA-based oligonucleotide has the advantages of increased hybridization affinity and enhanced resistance to nuclease degradation.

“Unlocked Nucleic Acid” or “UNA”, is as referred to herein unlocked nucleic acid typically where the C2-C3 C—C bond of the ribose has been removed, forming an unlocked “sugar” residue (see Fluiter et al., Mol. Biosyst., 2009, 10, 1039, hereby incorporated by reference, and Snead et al. Molecular Therapy—Nucleic Acids (2013) 2, e103).

“Target region” means a portion of a target nucleic acid to which one or more antisense compounds is targeted.

“Targeted delivery” as used herein means delivery, wherein the antisense oligonucleotide has either been formulated in a way that will facilitate efficient delivery in specific tissues or cells, or wherein the antisense oligonucleotide in other ways has been for example modified to comprise a targeting moiety, or in other way has been modified in order to facilitate uptake in specific target cells.

The antisense oligonucleotides of the invention are designed to target adenosine kinase (ADK)

The term “adenosine kinase related neurological disease” as used herein means diseases where disease pathology is linked with upregulation of adenosine kinase activity, or where downregulation of adenosine kinase activity will be beneficial for treatment of the disease.

### Compounds

The human ADK gene encodes 14 transcripts. Of these transcripts, 10 are protein coding and therefore potential nucleic acid targets. A number of ASOs were designed to target a 5′-region (SEQ ID NO 2) on the ADK pre-mRNA. Specifically SEQ ID NO 2 is the region from position 1 to position 25349 on the ADK pre mRNA. For example, the ASOs were constructed to target nucleotides 74-75, 132-134, 782, 830, 926, 5865, 7203, 15679, 24985, and 25284 of SEQ ID NO: 2.

The exemplary sequences of the ASOs are described in Table 1 and Table 2. In some embodiments, the ASOs were designed to be gapmers or mixmers. Gapmers will recruit RNAse H for cleavage of target RNA, whereas mixmers do not recruit RNAse H.

Tables 1 and 2 contain non-limiting examples of the ASO design for selected sequences. The same methods can be applied to any other sequences disclosed herein. The gapmers were constructed to contain locked nucleic acids—LNAs (upper case letters). For example, a gapmer can have Beta-deoxy LNA at the 5′ end and the 3′ end and have a phosphorothioate backbone. But the LNAs can also be substituted with any other nucleotide analog and the backbone can be another type of backbone {e.g., a phosphodiester linkage, a phosphotriester linkage, a methylphosphonate linkage, a phosphoramidate linkage, or combinations thereof).

In Tables 1 and 2, in the Compound designation, upper case designates a modified nucleotide such as an LNA nucleotide (either Beta-D-Oxy, Alpha-L-Oxy, Beta-D-Amino or Beta-D-Thio LNA or other modified nucleotide such as cEt, cMOE, UNA or ENA) and lower case designates a DNA nucleotide.

Thus a sequence represented by AAaatggccgcGCC (SEQ ID NO: 63) represents a 2-9-3 14mer modified nucleotide-DNA-modified nucleotide gapmer with a 5′-A and 3′-C, such as a 2-9-3 LNA-DNA-LNA gapmer. Some ASOs can be an alternating flank gapmer as described elsewhere herein. In some embodiments, selected examples of alternating flank gapmers having a 7 nucleotide gap are SEQ ID NOs 5, 33 and 71.

In its broadest sense, the invention provides antisense oligonucleotides complementary to adenosine kinase (ADK) RNA (SEQ ID NO: 1) comprising a sequence of 10-30 nucleotides in length, wherein the antisense oligonucleotide comprises at least one affinity-enhancing nucleotide analogue and wherein said antisense oligonucleotide comprises at least one phosphorothioate or similar internucleoside linkage. In some embodiments, the antisense oligonucleotides of the invention has an alternative to phosphorothioate internucleoside linkage, such as the backbone can be another type of backbone e.g., a phosphodiester linkage, a phosphotriester linkage, a methylphosphonate linkage, a phosphoramidate linkage, or combinations thereof. In preferred embodiments, an alternative nucleoside backbone is suitable for medical use of the antisense oligonucleotide.

In some embodiments, the antisense oligonucleotide according to the invention is complementary to SEQ ID NO:2 which is the 5′ end of SEQ ID NO: 1 (nucleotide 1:25349 of ADK pre mRNA) and which is specific for the mRNA that encodes the long isoform of ADK. Thus in some embodiments, the invention provides antisense oligonucleotides that selectively target mRNA that encodes the long isoform of ADK. In some embodiments, the antisense oligonucleotide according to the invention is complementary to SEQ ID NO: 120 or 121. In some embodiments, the antisense oligonucleotide according to the invention is a mixmer that is complementary to SEQ ID NO:120 or 121 and inhibit splicing of the ADK RNA. In some embodiments, the antisense oligonucleotide according to the invention is complementary to anyone of SEQ ID NO: 122-132. In some embodiments, the antisense oligonucleotide according to the invention comprises anyone of SEQ ID NO: 101-119. In some embodiments, the antisense oligonucleotide according to the invention is a mixmer, i.e. it does not contain a contiguous stretch of more than 4 DNA nucleotides, such as no more than 3 contiguous DNA nucleotides. In some embodiments, the antisense oligonucleotide according to the invention is a gapmer, wherein the antisense oligonucleotide contains a contiguous stretch of at least five contiguous DNA nucleotides. The size of an antisense oligonucleotide for medical purposes matters, thus the antisense oligonucleotides according to the present invention are designed to be useful for such use. In some embodiments, the antisense oligonucleotides according to the invention are 10-30 nucleotides in length, and in some embodiments, the antisense oligonucleotide is 14-19 nucleotides in length.

The efficacy of an antisense of an antisense oligonucleotide depend on stability, affinity towards the target RNA and other factors. Presence of affinity enhancing nucleoside analogues such as LNA in an antisense oligonucleotide provide such advantages. In preferred embodiments, the affinity-enhancing nucleotide analogues used in the antisense oligonucleotides of the present invention are selected from the list of LNA, tricyclo-DNA, 2′-Fluoro, 2′-O-methyl, 2′methoxyethyl (2′MOE), 2′ cyclic ethyl (CET), UNA, 2′fluoro and Conformationally Restricted Nucleoside (CRN). In some embodiments, such oligonucleotide may comprise a combination of LNA, DNA and one or more of tricyclo-DNA, 2′-Fluoro, 2′-O-methyl, 2′methoxyethyl (2′MOE), 2′ cyclic ethyl (CET), UNA, 2′fluoro and Conformationally Restricted Nucleoside (CRN).

In some embodiments, the antisense oligonucleotide according to the invention, comprises at least one LNA. In some embodiments, the antisense oligonucleotide comprises from 30-55% LNA. In some embodiments, the antisense oligonucleotide according to the invention is a LNA/DNA oligonucleotide, but further comprises one or more nucleosides that are anyone of tricyclo-DNA, 2′-Fluoro, 2′-O-methyl, 2′methoxyethyl (2′MOE), 2′ cyclic ethyl (CET), UNA, 2′fluoro and Conformationally Restricted Nucleoside (CRN).

In some preferred embodiments, the antisense oligonucleotide according to the invention comprises LNA, wherein the LNA is Beta-D-Oxy LNA.

In some embodiments, the antisense oligonucleotide according to the invention is designed so that all the internucleoside bonds are phosphorothioate bonds. In some embodiments, the present invention provides a series of potent antisense oligonucleotides, wherein the antisense oligonucleotide is anyone of SEQ ID NO's 3-73. In some embodiments, the present invention provides a series of potent antisense oligonucleotides, wherein the antisense oligonucleotide is anyone of SEQ ID NO's 74-100. In some embodiments, the present invention provides a series of potent antisense oligonucleotides, wherein the antisense oligonucleotide is anyone of SEQ ID NO's 133-147. In some embodiments, the present invention provides an antisense oligonucleotide selected from SEQ ID NO's: 10, 11, 14, 16, 18, 23, 36, 41, 42, 43, 64, 65, 133, 134, 135, 136, 137, 138, 139, 140, 141, 142, 143, 144, 145, 146, or 147.

In Tables 1 and 2 the listed ASOs are always depicted in the 5′ to 3′ direction. Therefore, the 5′ end of an ASO hybridizes to the pre-mRNA “end” number in the table and the 3′ end of the ASO hybridizes to the pre-mRNA “start” number in the tables.

Table 1 provide a list of motifs for antisense oligonucleotides that are complementary to SEQ ID NO: 1). Table 1 further present ASO gapmer designs comprising the motifs sequences. Table 2 provide a list of motifs for antisense oligonucleotides that are complementary to SEQ ID NO: 1). Table 2 further present ASO mixmer designs comprising the motif sequences.

In some embodiments, the antisense oligonucleotide according to the invention is complementary to a sequence within SEQ ID NO: 2, which is the sequence that is unique in the ADK long form. In some embodiments, LNA mixmers are designed that target the ADK pre mRNA near the splicing site, and prevent the splicing event. In some embodiments, mixmers oligonucleotides are complementary to a sequence within SEQ ID NO: 120; 5′ GATGCGAAGA GGGGGGGGGA CCAGAGAGTG GATGGCAGAG GTGGGCTGTA GAGCCAAAGT GGGGTGGGAG CGCGAAGATG GCAGCTGCTG AGGAGGAGCC GAAGCCCAAA AAGCTGAAGG TGGAGGCGCC GCAAGCGCTG AGGTGAGCGC TGCCGGACTT GGGGAGGAGG GTGACGGCGC TGCAAGCAAG CCAGGGCCCA CGTGGGGTTG CACGGCCCCG ACGCTGGGTG GTGTCTCTCA CTGCCAGCTT 3′.

In some embodiments, the mixmers of the invention are complementary to a sequence within SEQ ID NO: 121; 5′ GAAGATGGCA GCTGCTGAGG AGGAGCCGAA GCCCAAAAAG CTGAAGGTGG AGGCGCCGCA AGCGCTGAGG TGAG 3′.

In some embodiments, the antisense oligonucleotides of the invention are complementary to anyone of SEQ ID NO: 122-132.

Table 3 specific ASO target sequences.

Table 1 lists position of target regions in SEQ ID NO: 1 (complete ADK pre mRNA sequence), motifs of individual antisense oligonucleotides of the invention (SEQ ID NO: 101-111) and specific antisense oligonucleotide gapmer compounds (SEQ ID NO: 3-73 and 133-147).

Table 2 lists position of target regions in SEQ ID NO: 1, motifs of individual antisense oligonucleotides of the invention (SEQ ID NO: 112-119) and specific antisense oligonucleotide mixmer compounds (SEQ ID NO: 74-100).

TABLE 1
List of gapmers or contiguous nucleobase sequences complementary to SEQ ID NO: 1), ASO
designs made from these, as well as specific ASOs compounds designed based on the motif
sequence. For SEQ ID NO′s: 3-74 and 133-147, upper case letters are affinity enhancing
nucleoside analogues, such as LNA, such as beta-d-oxy LNA, upper case C is LNA 5′methyl C,
and at least one internucleoside bond is a non-natural linkage such as a phosphorothioate
linkage.

| start SEQ | end SEQ | SEQ | | SEQ | |
|---|---|---|---|---|---|
| ID 1 | ID 1 | ID NO | motif | ID NO | compound |
| 132 | 146 | 101 | TCACCTCAGCGCTTG | 3 | TCacctcagcGctTG |
| 132 | 146 | 101 | TCACCTCAGCGCTTG | 4 | TCacctcAgcgctTG |
| 132 | 146 | 101 | TCACCTCAGCGCTTG | 5 | TCaccTcagcgctTG |
| 782 | 798 | 102 | AAAGTTGCAACATCAAT | 6 | AAagtTGCaacatCAAT |
| 782 | 798 | 102 | AAAGTTGCAACATCAAT | 7 | AAAgttGCaacatCAAT |
| 782 | 798 | 102 | AAAGTTGCAACATCAAT | 8 | AAaGTTgcaacatCAAT |
| 830 | 848 | 103 | CTTTATACTTATTAGGAAG | 9 | CTTtatacTtattagGAAG |
| 830 | 848 | 103 | CTTTATACTTATTAGGAAG | 10 | CTttatacttATtAGgAAG |
| 830 | 848 | 103 | CTTTATACTTATTAGGAAG | 11 | CTttAtacttattAGgAAG |
| 830 | 848 | 103 | CTTTATACTTATTAGGAAG | 12 | CTtTaTacttaTtaGgAAG |
| 830 | 848 | 103 | CTTTATACTTATTAGGAAG | 13 | CTTtAtacttAttaGgAAG |
| 830 | 848 | 103 | CTTTATACTTATTAGGAAG | 14 | CTTtatacttAtTAggAAG |
| 830 | 848 | 103 | CTTTATACTTATTAGGAAG | 15 | CTtTatacttATTaggAAG |
| 830 | 848 | 103 | CTTTATACTTATTAGGAAG | 16 | CTttatactTaTTaggAAG |
| 830 | 848 | 103 | CTTTATACTTATTAGGAAG | 17 | CTtTataCTtAttaggAAG |
| 830 | 848 | 103 | CTTTATACTTATTAGGAAG | 18 | CTttataCttatTAgGaAG |
| 830 | 848 | 103 | CTTTATACTTATTAGGAAG | 19 | CTttATacttatTAggaAG |
| 926 | 943 | 104 | GTTTTAAATCAGTTTGAT | 20 | GTtttaaAtCagTtTGAT |
| 926 | 943 | 104 | GTTTTAAATCAGTTTGAT | 21 | GTtttaaaTcagTtTGAT |
| 926 | 943 | 104 | GTTTTAAATCAGTTTGAT | 22 | GTtTtAaatcagTtTGAT |
| 926 | 943 | 104 | GTTTTAAATCAGTTTGAT | 23 | GTTtTaaatcagTtTGAT |
| 926 | 943 | 104 | GTTTTAAATCAGTTTGAT | 24 | GTtttaaatcAGttTGAT |
| 926 | 943 | 104 | GTTTTAAATCAGTTTGAT | 25 | GTtttaaAtCaGttTGAT |
| 926 | 943 | 104 | GTTTTAAATCAGTTTGAT | 26 | GTtTtaaatCaGttTGAT |
| 926 | 943 | 104 | GTTTTAAATCAGTTTGAT | 27 | GTTttAaatcaGttTGAT |
| 926 | 943 | 104 | GTTTTAAATCAGTTTGAT | 28 | GTtTTaaatcaGttTGAT |
| 926 | 943 | 104 | GTTTTAAATCAGTTTGAT | 29 | GTtttaaatcaGttTGAT |
| 926 | 943 | 104 | GTTTTAAATCAGTTTGAT | 30 | GTtttaaatcAgttTGAT |
| 5865 | 5881 | 105 | GCTTTTTAAAGCAACAG | 31 | GCtTTtTaaagcAaCAG |
| 5865 | 5881 | 105 | GCTTTTTAAAGCAACAG | 32 | GCtttttAAagCaaCAG |
| 5865 | 5881 | 105 | GCTTTTTAAAGCAACAG | 33 | GCtttttaaAGcaacAG |
| 5865 | 5881 | 105 | GCTTTTTAAAGCAACAG | 34 | GCtTttTaAAgcaacAG |
| 5865 | 5881 | 105 | GCTTTTTAAAGCAACAG | 35 | GCttttTaAAgcaacAG |
| 5865 | 5881 | 105 | GCTTTTTAAAGCAACAG | 36 | GCttTtTAAagcaacAG |
| 5865 | 5881 | 105 | GCTTTTTAAAGCAACAG | 37 | GCTttTTaaagcaacAG |
| 5865 | 5882 | 106 | AGCTTTTTAAAGCAACAG | 38 | AGctttttAaAgcAaCAG |
| 5865 | 5882 | 106 | AGCTTTTTAAAGCAACAG | 39 | AGctttttaaAgcAaCAG |
| 5865 | 5882 | 106 | AGCTTTTTAAAGCAACAG | 40 | AGcttTTtaaagcAaCAG |
| 5865 | 5882 | 106 | AGCTTTTTAAAGCAACAG | 41 | AGctTtTtAAagcaaCAG |
| 7203 | 7219 | 107 | CTTTGGGATTTCAGAAA | 42 | CTTtgggattTCaGAAA |
| 7203 | 7219 | 107 | CTTTGGGATTTCAGAAA | 43 | CTttgggattTCaGAAA |
| 7203 | 7219 | 107 | CTTTGGGATTTCAGAAA | 44 | CTttgggATttCaGAAA |
| 7203 | 7219 | 107 | CTTTGGGATTTCAGAAA | 45 | CTtTgGgatttCaGAAA |
| 7203 | 7219 | 107 | CTTTGGGATTTCAGAAA | 46 | CTttgGgatttCaGAAA |
| 7203 | 7219 | 107 | CTTTGGGATTTCAGAAA | 47 | CTtTgggatTTcaGAAA |
| 7203 | 7219 | 107 | CTTTGGGATTTCAGAAA | 48 | CTttgggATtTcaGAAA |
| 7203 | 7219 | 107 | CTTTGGGATTTCAGAAA | 49 | CTttgggATTtcaGAAA |
| 7203 | 7219 | 107 | CTTTGGGATTTCAGAAA | 50 | CTTtgggaTTtcaGAAA |
| 15679 | 15695 | 108 | CAAAGCTCTTTTCCTTG | 51 | CAaagctctTTtccTTG |
| 15679 | 15695 | 108 | CAAAGCTCTTTTCCTTG | 52 | CAaagctcTtTtccTTG |
| 15679 | 15695 | 108 | CAAAGCTCTTTTCCTTG | 53 | CAaAgctcttTtccTTG |
| 15679 | 15695 | 108 | CAAAGCTCTTTTCCTTG | 54 | CAAagctcttTtccTTG |
| 15679 | 15695 | 108 | CAAAGCTCTTTTCCTTG | 55 | CAaagctcttTtccTTG |
| 15679 | 15695 | 108 | CAAAGCTCTTTTCCTTG | 56 | CAaagctcTtttccTTG |
| 24985 | 25001 | 109 | AACTTCAGAAACACTTA | 57 | AACttCAgaaacaCTTA |
| 24985 | 25001 | 109 | AACTTCAGAAACACTTA | 58 | AACtTCAgaaacaCtTA |
| 24985 | 25002 | 110 | GAACTTCAGAAACACTTA | 59 | GAActtcagAaAcaCTTA |
| 24985 | 25002 | 110 | GAACTTCAGAAACACTTA | 60 | GAaCttcAgaaacaCTTA |
| 24985 | 25002 | 110 | GAACTTCAGAAACACTTA | 61 | GAacTtCAgaaacAcTTA |
| 24985 | 25002 | 110 | GAACTTCAGAAACACTTA | 62 | GAActtcagaAACacTTA |
| 24985 | 25002 | 110 | GAACTTCAGAAACACTTA | 63 | GAacttcAGaAaCacTTA |
| 24985 | 25002 | 110 | GAACTTCAGAAACACTTA | 64 | GAaCttCagaaaCacTTA |
| 24985 | 25002 | 110 | GAACTTCAGAAACACTTA | 65 | GAACttcagaaaCacTTA |
| 24985 | 25002 | 110 | GAACTTCAGAAACACTTA | 66 | GAActtCAgAaacacTTA |
| 24985 | 25002 | 110 | GAACTTCAGAAACACTTA | 67 | GAactTcagaaACACtTA |
| 25284 | 25297 | 111 | AAAATGGCCGCGCC | 68 | AAaatggccgcGCC |
| 25284 | 25297 | 111 | AAAATGGCCGCGCC | 69 | AAaatggcCgCgCC |
| 25284 | 25297 | 111 | AAAATGGCCGCGCC | 70 | AAaaTggccgCgCC |
| 25284 | 25297 | 111 | AAAATGGCCGCGCC | 71 | AAAatggccgCgCC |
| 25284 | 25297 | 111 | AAAATGGCCGCGCC | 72 | AAaatggccgCgCC |
| 25284 | 25297 | 111 | AAAATGGCCGCGCC | 73 | AAAatggccGcgCC |
| 5865 | 5882 | 106 | AGCTTTTTAAAGCAACAG | 133 | AGctttTtAAagcaaCAG |
| 5865 | 5882 | 106 | AGCTTTTTAAAGCAACAG | 134 | AGctTtttAAagcaaCAG |
| 5865 | 5882 | 106 | AGCTTTTTAAAGCAACAG | 135 | AGctTtTtAaagcaaCAG |
| 5865 | 5882 | 106 | CTTTATACTTATTAGGAAG | 136 | CTttatactTaTTaGgAAG |
| 5865 | 5882 | 106 | CTTTATACTTATTAGGAAG | 137 | CTttatacttATtaGgAAG |
| 5865 | 5882 | 106 | CTTTATACTTATTAGGAAG | 138 | CTttatacttATtAggAAG |
| 7203 | 7219 | 107 | CTTTGGGATTTCAGAAA | 139 | CTttgggatTTCaGAAA |
| 7203 | 7219 | 107 | CTTTGGGATTTCAGAAA | 140 | CTTtgggattTcaGAAA |
| 7203 | 7219 | 107 | CTTTGGGATTTCAGAAA | 141 | CTTtgggattTCagAAA |
| 24985 | 25002 | 110 | GAACTTCAGAAACACTTA | 142 | GAACttcagaaACacTTA |
| 24985 | 25002 | 110 | GAACTTCAGAAACACTTA | 143 | GAACttcagaaaCactTA |
| 5865 | 5881 | 105 | GCTTTTTAAAGCAACAG | 144 | GCttTtTAAagcaaCAG |
| 5865 | 5881 | 105 | GCTTTTTAAAGCAACAG | 145 | GCtTTtTAAagcaacAG |
| 926 | 943 | 104 | GTTTTAAATCAGTTTGAT | 146 | GTttTaaatcagTtTGAT |
| 926 | 943 | 104 | GTTTTAAATCAGTTTGAT | 147 | GTTtTaaatcagTttGAT |

TABLE 2
List of mixmers or contiguous nucleobase sequences complementary to SEQ ID NO: 1),
ASO designs made from these, as well as specific ASOs compounds designed based on the motif
sequence. For SEQ ID NO′s: 74-100, upper case letters are affinity enhancing nucleoside
analogues, such as LNA, such as beta-d-oxy LNA, upper case C is LNA 5′methyl C, and at least
one internucleoside bond is a non-natural linkage such as a phosphorothioate linkage.

| start | end | Motif | | Compound | |
|---|---|---|---|---|---|
| SEQ | SEQ | SEQ ID | | SEQ ID | |
| ID 1 | ID 1 | NO: | motif | NO: | compound |
| 74 | 87 | 112 | CAGCTGCCATCTTC | 74 | CAGctGccaTCTTC |
| 74 | 87 | 112 | CAGCTGCCATCTTC | 75 | CAgcTgCcatCtTC |
| 74 | 87 | 112 | CAGCTGCCATCTTC | 76 | CAgctGccAtctTC |
| 74 | 88 | 113 | GCAGCTGCCATCTTC | 77 | GCagcTgCcaTctTC |
| 74 | 88 | 113 | GCAGCTGCCATCTTC | 78 | GCaGctGccaTctTC |
| 74 | 88 | 113 | GCAGCTGCCATCTTC | 79 | GCagcTgccAtctTC |
| 75 | 88 | 114 | GCAGCTGCCATCTT | 80 | GCAgctGccAtCTT |
| 75 | 88 | 114 | GCAGCTGCCATCTT | 81 | GCagcTgccAtcTT |
| 75 | 88 | 114 | GCAGCTGCCATCTT | 82 | GCaGcTgcCatcTT |
| 132 | 145 | 115 | CACCTCAGCGCTTG | 83 | CACcTCaGcgcTTG |
| 132 | 145 | 115 | CACCTCAGCGCTTG | 84 | CAcctCagCGctTG |
| 132 | 145 | 115 | CACCTCAGCGCTTG | 85 | CAcctCagcGctTG |
| 132 | 146 | 101 | TCACCTCAGCGCTTG | 86 | TCacCTcAgcGcTTG |
| 132 | 146 | 101 | TCACCTCAGCGCTTG | 87 | TCacCtcAgcGcTTG |
| 132 | 146 | 101 | TCACCTCAGCGCTTG | 88 | TCaccTcAgcGctTG |
| 132 | 147 | 116 | CTCACCTCAGCGCTTG | 89 | CTCAccTcAgcGctTG |
| 132 | 147 | 116 | CTCACCTCAGCGCTTG | 90 | CTcaCctCagcGctTG |
| 132 | 147 | 116 | CTCACCTCAGCGCTTG | 91 | CTcAcctCagcGctTG |
| 133 | 146 | 117 | TCACCTCAGCGCTT | 92 | TCaccTCagcGCTT |
| 133 | 146 | 117 | TCACCTCAGCGCTT | 93 | TCAcctCaGcGcTT |
| 133 | 146 | 117 | TCACCTCAGCGCTT | 94 | TCaccTcAgcGcTT |
| 133 | 147 | 118 | CTCACCTCAGCGCTT | 95 | CTcAcctCagcGcTT |
| 133 | 147 | 118 | CTCACCTCAGCGCTT | 96 | CTCAccTcAgCgcTT |
| 133 | 147 | 118 | CTCACCTCAGCGCTT | 97 | CTcACctcAGcgcTT |
| 134 | 147 | 119 | CTCACCTCAGCGCT | 98 | CTcAcCTcagCgCT |
| 134 | 147 | 119 | CTCACCTCAGCGCT | 99 | CTcaCcTcaGcgCT |
| 134 | 147 | 119 | CTCACCTCAGCGCT | 100 | CTcAccTcAgcgCT |

TABLE 3
target sequences for the ASOs of Table 1.

| SEQ ID NO: | Target sequence |
|---|---|
| 122 | CAAGCGCTGAGGTGA |
| 123 | ATTGATGTTGCAACTTT |
| 124 | CTTCCTAATAAGTATAAAG |
| 125 | ATCAAACTGATTTAAAAC |
| 126 | CTGTTGCTTTAAAAAGC |
| 127 | CTGTTGCTTTAAAAAGCT |
| 128 | TTTCTGAAATCCCAAAG |
| 129 | CAAGGAAAAGAGCTTTG |
| 130 | TAAGTGTTTCTGAAGTT |
| 131 | TAAGTGTTTCTGAAGTTC |
| 132 | GGCGCGGCCATTTT |

In some embodiments, the antisense oligonucleotide of the invention is anyone of SEQ ID NO's: 3-73, or 74-100 or 133 to 147, wherein the oligonucleotide comprise at least one non natural internucleoside linkage, such as a phosphorothioate linkage. In some embodiments, the oligonucleotide of the invention is anyone of SEQ ID NO's: 3-73, or 74-100 or 133 to 147, and comprise at least 5 non natural internucleoside linkages, such as at least 5 phosphorothioate linkages. In some embodiments, the oligonucleotide of the invention is anyone of SEQ ID NO's: 3-73, or 74-100 or 133 to 147, and comprise at least 7, such as at least 8 or at least 9 or at least 10 or at least 15 or all internucleoside linkages are non natural, such as phosphorothioate linkages. In some embodiments, the oligonucleotide of the invention is anyone of SEQ ID NO's: 3-73, or 74-100 or 133 to 147, and non natural nucleosides are LNA.

In some embodiments, the oligonucleotide of the invention is anyone of SEQ ID NO's: 3-73, or 74-100 or 133 to 147, and non natural nucleosides are LNA, such as beta-d-oxy LNA and LNA C is 5′methyl C. In some embodiments, the oligonucleotide of the invention is anyone of SEQ ID NO's: 3-73, or 74-100 or 133 to 147, and non natural nucleosides are beta-d-oxy LNA and LNA C is 5′methyl C and all internucleoside linkages are phosphorothioate linkages.

In some embodiments, the oligonucleotide of the invention is selected from the list of SEQ ID NO's: 10, 11, 14, 16, 18, 23, 36, 41, 42, 43, 64, 65, 133, 134, 135, 136, 137, 138, 139, 140, 141, 142, 143, 144, 145, 146, or 147. In some embodiments, the oligonucleotide of the invention is selected from the list of SEQ ID NO's: 10, 11, 14, 16, 18, 23, 36, 41, 42, 43, 64, 65, 133, 134, 135, 136, 137, 138, 139, 140, 141, 142, 143, 144, 145, 146, or 147, wherein according to the listings in Table 1 and 2, upper case letters are LNA, such as beta-d-oxy LNA, LNA C is 5′methyl C, and all internucleoside bonds are phosphorothioate linkages.

In some embodiments, the antisense oligonucleotide according to the invention is anyone of:
oligonucleotide is anyone of SEQ ID NO's:

(SEQ ID NO 10)
5′ CTttatacttATtAGgAAG 3′
or
(SEQ ID NO 16)
5′ CTttatactTaTTaggAAG 3′
or
(SEQ ID NO 41)
5′ AGctTtTtAAagcaaCAG 3′
or
(SEQ ID NO 42)
5′ CTTtgggattTCaGAAA 3′
or
(SEQ ID NO 43)
5′ CTttgggattTCaGAAA 3′
or
(SEQ ID NO 136)
5′ CTttatactTaTTaGgAAG 3′
or
(SEQ ID NO 137)
5′ CTttatacttATtaGgAAG 3′
or
(SEQ ID NO 139)
5′ CTttgggatTTCaGAAA 3′

Wherein upper case letters are LNA, such as beta-d-oxy LNA, LNA C is 5′methyl C, and all internucleoside bonds are phosphorothioate linkages.

In some embodiments, the invention provides RNA inhibitory agents such as an siRNA that target ADK RNA (SEQ ID NO: 1). In some embodiments, the invention provides an siRNA that selectively target mRNA encoding the long isoform of ADK (SEQ ID NO: 2). In some embodiments, the invention provides an siRNA that target ADK-L mRNA and comprise any one of SEQ ID NO's: 3-73 or 74-100 or 133-147. In some embodiments, the siRNA of the invention comprise any one of SEQ ID NO's: 101-119. In some embodiments, the target sequence of the siRNA of the invention comprise the RNA sequence corresponding to any one of SEQ ID NO's: 122-132.

siRNA's are well known in the art, and can easily be designed by a skilled person as described in WO16/057893 (included by reference).

### Compositions and Uses

The compounds of the invention are for use in the compositions, such as in the pharmaceutical compositions of the invention, and for the use as medicaments, and for treatment, alleviation, amelioration, pre-emptive treatment, or prophylaxis of the diseases disclosed herein, such as neurological disorders, including epilepsy.

The compounds of the invention are in some embodiments comprised in compositions, such as pharmaceutical compositions for treatment of diseases, which are diseases where modulation of adenosine kinase activity is beneficial for preventive, curative or disease modifying treatment, prophylaxis, alleviation or amelioration of the disease or disease parameters. In some embodiments, the treatment, prophylaxis, alleviation or amelioration is curative. In some embodiments, the treatment, prophylaxis, alleviation or amelioration is disease modifying. In some embodiments, the treatment, prophylaxis, alleviation or amelioration is preventive.

Diseases that may be treated, alleviated, ameliorated, pre-emptively treated or prophylactically treated by the compounds and compositions include in non-limiting example diseases of the central nervous system (CNS) or peripheral nervous system (PNS), including neurological disorders, neurodegenerative disorders, neurodevelopmental disorders, or psychiatric diseases. In some embodiments, the antisense oligonucleotide or composition according to the invention is for use as a neuroprotective agent.

In some embodiments, the antisense oligonucleotide or composition according to the invention is for use in the treatment, alleviation, pre-emptive treatment or prophylaxis of a disease of the CNS or PNS, a neurological disorder, a neurodegenerative disorder, a neurodevelopmental disorder, a central and peripheral nervous system diseases associated with cellular trauma and inflammation, neuronal damage, hippocampal damage, traumatic brain injury, a memory disorder, hippocampal sclerosis, Parkinsons Disease, multiple sclerosis, acute spinal cord injury, amyotrophic lateral sclerosis, ataxia, bell's palsy, Charcot-Marie-Tooth, a headache, Horton's headache, migraine, pick's disease, progressive supranuclear palsy, multi-system degeneration, a motor neuron disease, Huntington's disease, prion disease, Creutzfeldt-Jakob disease, corticobasal degeneration, primary progressive aphasia or symptoms or effects thereof.

In some embodiments, the antisense oligonucleotide or composition according to the invention is for use in the treatment of epilepsy.

In some embodiments, the antisense oligonucleotide or composition according to the invention is for use in the treatment of seizures.

In some embodiments, the antisense oligonucleotide or composition according to the invention is for use in the treatment, alleviation, pre-emptive treatment or prophylaxis of epilepsy and/or seizures, preferably a treatment resistant epilepsy, acquired, genetic and/or idiopathic epilepsy, therapy resistant epileptic syndromes, drug resistant epilepsy, pharmacy resistant focal epilepsy, spontaneous seizures, therapy resistant seizures, focal epilepsy, generalised epilepsy or status epilepticus.

In some embodiments, the antisense oligonucleotide or composition according to the invention is for use in the treatment, alleviation, pre-emptive treatment or prophylaxis of epilepsy, drug resistant epilepsy, pharmacoresistant focal epilepsy, seizures, spontaneous seizures, therapy resistant seizures, focal epilepsy, preferably wherein said focal epilepsy is focused in the frontal lobe, the parietal lobe, the occipital lobe or the temporal lobe, generalised epilepsy, preferably wherein said generalised epilepsy is selected among absences, myoclonic seizures, tonic-clonic seizures, tonic seizures, atonic seizures, clonic seizures and spasms, status epilepticus, epileptogenesis induced by acute brain injury, autosomal dominant nocturnal frontal lobe epilepsy, continuous spike-and-waves during slow sleep, dravet syndrome, epilepsy developed after apoplexy, epileptic encephalopathy, gelastic epilepsy, absences, benign neonatal seizures, Jeavons syndrome, Juvenile myoclonic epilepsy, Landau-Kleffner Syndrom, Lennox-Gastaut syndrome, Mesial temporal lobe epilepsy, myoclonic astatic epilepsy, Ohtahara Syndrom, Panayiotopoulos syndrome, PCDH19 syndrom, benign childhood epilepsy with centrotemporal spikes, Sturge-Weber syndrome, symptomatic focal epilepsy, transient epileptic amnesia and West syndrome, and/or glioma-associated epilepsy.

In some embodiments, the antisense oligonucleotide or composition according to the invention is for use in the treatment, alleviation, pre-emptive treatment or prophylaxis of pain, preferably wherein said pain is a chronic pain, a neuropathic pain, a chemotherapy-induced neuropathic pain, a migraine, a headaches, hyperalgesia, allodynia and/or fibromyalgia.

In some embodiments, the antisense oligonucleotide or composition according to the invention is for use in the treatment of pain.

In some embodiments, the antisense oligonucleotide or composition according to the invention is for use in the treatment, alleviation, pre-emptive treatment or prophylaxis of pain, chronic plain, neuropathic pain, chemotherapy-induced neuropathic pain, migraine, including migraine with aura and migraine without aura, a primary headache, a tension headache, a cluster headache, Hortons headache, a chronic daily headache, a sinus headache, a posttraumatic headache, an exercise headache, hemicrannia continua, hypnic headache, hyperalgesia, thermal hyperalgesia, allodynia, tactile allodynia and/or fibromyalgia.

In some embodiments, the antisense oligonucleotide or composition according to the invention is for use in the treatment, alleviation, pre-emptive treatment or prophylaxis of a psychiatric disorder, a cognitive disorder, a sleep disorder, a cardiovascular disorder, a respiratory disorder, a cancer, a renal disorder, an inflammation or a metabolic disorder.

In some embodiments, the antisense oligonucleotide or composition according to the invention is for use in the treatment, alleviation, pre-emptive treatment or prophylaxis of a psychiatric disorder, a neuropsychiatric disorder, anxiety, depression, bipolar disorder, attention deficit hyperactive disorder, attention deficit disorder, autism, Asperger's, Tourette, schizophrenia, paranoid schizophrenia, hebephrenic schizophrenia, catatonic schizophrenia, undifferentiated schizophrenia, residual schizophrenia, simple schizophrenia or unspecified schizophrenia.

In some embodiments, the antisense oligonucleotide or composition according to the invention is for use in the treatment, alleviation, pre-emptive treatment or prophylaxis of a cognitive disorder, cognitive impairment, dementia, Alzheimer disease, vascular dementia, frontotemporal dementia or Lewy bodies dementia.

In some embodiments, the antisense oligonucleotide or composition according to the invention is for use in the treatment, alleviation, pre-emptive treatment or prophylaxis of a sleep disorder.

In some embodiments, the antisense oligonucleotide or composition according to the invention is for use as a sleep modulating agent.

In some embodiments, the antisense oligonucleotide or composition according to the invention is for use in sleep promotion.

In some embodiments, the antisense oligonucleotide or composition according to the invention is for use in the treatment, alleviation, pre-emptive treatment or prophylaxis of a cardiovascular disorders, a peripheral artery disease, postoperative atrial fibrillation, heart failure, chronic heart failure, intracerebral haemorrhage-induced brain injury, stroke, cerebral ischemia or ischaemia.

In some embodiments, the antisense oligonucleotide or composition according to the invention is for use in the treatment, alleviation, pre-emptive treatment or prophylaxis of a respiratory disorder, asthma or chronic obstructive pulmonary disease.

In some embodiments, the antisense oligonucleotide or composition according to the invention is for use in the treatment, alleviation, pre-emptive treatment or prophylaxis of a cancer, a cancer in the nerve system, glioma, glioblastoma, hepatic cancer or a cancer metastasis.

In some embodiments, the antisense oligonucleotide or composition according to the invention is for use in the treatment, alleviation, pre-emptive treatment or prophylaxis of a renal disorder, renal injury, renal inflammation, albuminuria or glomerular injury.

In some embodiments, the antisense oligonucleotide or composition according to the invention is for use in the treatment, alleviation, pre-emptive treatment or prophylaxis of inflammation.

In some embodiments, the antisense oligonucleotide or composition according to the invention is for use in the treatment, alleviation, pre-emptive treatment or prophylaxis of an inflammatory disorder, oxidative stress, inflammation, apoptosis, arthritis, osteoarthritis, rheumatoid arthritis, and the pain associated with these conditions, encephalitis, meningitis, human Rasmussen encephalitis, inflammation of cerebral cortex and/or hippocampus, progressive cognitive deterioration, colitis, ulcerative colitis or inflammatory bowel disease.

In some embodiments, the antisense oligonucleotide or composition according to the invention is for use in the treatment, alleviation, pre-emptive treatment or prophylaxis of a metabolic disorder, preferably diabetes, more preferably type 1 or type 2 diabetes.

In some embodiments, the antisense oligonucleotide or composition according to the invention is for use in the treatment, alleviation, pre-emptive treatment or prophylaxis of Prader-Willis Syndrome, Anglemans Syndrome, neurofibromatosis, an angiogenesis related disease, promotion of angiogenesis, a disorder of the retina, preferably diabetic retinopathy or hearing loss.

In some embodiments, the antisense oligonucleotide or composition according to the invention is administered by systemic administration, intrathecal administration, intraventricular administration into the CNS or intravenous administration.

In some embodiments, the antisense oligonucleotide or composition according to the invention is for use in combination with one or more other active pharmaceutical ingredients for the treatment of anyone of the diseases of the invention.

According to an embodiment, the invention concerns the use of the antisense oligonucleotides according to the invention, wherein the other active pharmaceutical ingredient is an ingredient made for treatment of the diseases of the invention.

According to an embodiment, the invention concerns the use of the antisense oligonucleotides according to the invention, wherein the other pharmaceutical ingredient is an antisense oligonucleotide targeting miR-27b or miR-134 or both.

According to an embodiment, the invention concerns a pharmaceutical composition comprising an effective dosage of the antisense oligonucleotide according to the invention and a pharmaceutically acceptable carrier.

According to an embodiment, the invention concerns a pharmaceutical composition comprising an effective dosage of the antisense oligonucleotide according to the invention, wherein said antisense oligonucleotide is the sole active pharmaceutical ingredient.

In some embodiments, the anti adenosine kinase compounds may advantageously be used together with other therapies for a certain disease to be treated by the anti adenosine kinase composition.

Thereby, the anti adenosine kinase antisense oligonucleotide of the invention is for use in combination with one or more other therapies. In some embodiments, said other therapy is an anti miR-27b antisense oligonucleotide. In some embodiments, said other therapy is an anti miR-134 antisense oligonucleotide. In some embodiments, said other therapy induces the Nrf-2/ARE pathway in a mammal, such as in a human. In some embodiments, the anti adenosine kinase antisense oligonucleotide compositions are to be used in combination with one or more of an anti miR27b antisense oligonucleotide, an anti miR-134 antisense oligonucleotide and a therapy inducing the Nrf-2/ARE pathway.

In some embodiments, the antisense oligonucleotide targeting adenosine kinase of the invention are to be used in compositions where they are the sole active ingredient, and in some embodiments, they are for use in compositions comprising other active pharmaceutical ingredients.

The invention provides pharmaceutical compositions comprising the anti andenosine kinase antisense oligonucleotide compounds of the invention further comprising a pharmaceutically acceptable carrier.

In some embodiments, the pharmaceutical compositions of the invention comprises the anti adenosine kinase antisense oligonucleotide as the sole active pharmaceutical ingredient. In some embodiments, one or more active pharmaceutical ingredients are present in the pharmaceutical compositions of the invention.

### Dosages

The expression “effective dosage” denotes the dose of a drug that will achieve the desired effect. In the context of the present invention, the desired effect is lowering of the activity of adenosine kinase. Lowering of the activity of adenosine kinase can be measured by either measuring the level of adenosine kinase, for example when using oligonucleotides which result in degradation of ADK mRNA or ADK pre mRNA.

The compounds of the invention are for use in effective dosages, and the compositions comprise effective dosages of the compounds of the invention.

In some embodiments, the dosage of the compound administered at each dosing, such as unit dose, is within the range of 0.001 mg/kg-25 mg/kg.

In some embodiments, the effective dose is a dose that is sufficient to down-regulate adenosine kinase or the activity thereof, to a significant level over the time period between successive administration dosages, such as a level which is a therapeutic benefit to the subject.

The pharmaceutical compositions of the invention may in some embodiments be made for administration to provide for an initial dosage build up phase, which may, depending on the disease pathology, be followed by a maintenance dosage scheme for the purpose of maintaining a concentration of the compound in the subject, such as in a target tissue of the subject, which will be effective in the treatment of the disease. The effectiveness of the dosages may in example be measured by observation of a disease parameter indicative of the state of the disease, or may depending on the target tissue, be measurable by observation of various tissue parameters, such as activity of adenosine kinase, or in alternative example on a measurable disease state dependent parameter in plasma.

### Drug Delivery

Various delivery systems are known and can be used to administer a therapeutic of the invention. Methods of administration includes but are not limited to subcutaneous administration, intravenous administration, parenteral administration, nasal administration, pulmonary administration, rectal administration, vaginal administration, intrauterine administration, Intraurethral administration, administration to the eye, administration to the ear, cutaneous administration, intradermal administration, intramuscular administration, intraperitoneal administration, epidural administration, intraventricular administration, intracerebral, intrathecal administration or oral administration or administration directly into the brain or cerebrospinal fluid. The compositions may be administered by any convenient route, for example by infusion or bolus injection, by absorption through epithelial or mucocutaneous tissue (e.g., oral mucosa, rectal and intestinal mucosa, etc.) and may be administered together with or without other biologically active agents. Administration can be systemic or local. In addition, it may be desirable to administer the compositions of the invention into the central nervous system by any suitable route, including intraventricular and intrathecal administration. Intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. Preferably, the therapeutic is delivered to the CNS or PNS.

Delivery means include inhaled delivery, intramuscular delivery directly into a muscle by syringe or mini osmotic pump, intraperitoneal administration directly administered to the peritoneum by syringe or mini osmotic pump, subcutaneous administration directly administered below the skin by syringe, intraventricular administration direct administration to the ventricles in the brain, by injection or using small catheter attached to an osmotic pump. Further, an implant can be prepared (e.g. small silicon implant) that will be placed in a muscles or directly onto the spinal cord. It may be desirable to administer the compositions of the invention locally to the area in need of treatment; this may be achieved for example and not by way of limitation, by topical application, by injection, by means of a catheter, by means of a suppository, or by means of an implant, said implant may be of a porous, non-porous, or gelatinous material, including membranes, such as sialastic membranes, or fibers.

### Pharmaceutical Compositions

The present invention also provides pharmaceutical compositions. Such compositions may comprise a therapeutically effective amount of the therapeutic, and a pharmaceutically acceptable carrier. The term “pharmaceutically acceptable” may be defined as approved by a regulatory agency. The regulatory agency may for example be the European Medicines Agency, a Federal or a state government or listed in the U.S. Pharmacopeia or other generally recognized pharmacopeia for use in animals, and more particularly in humans. The term “therapeutically effective amount” may be defined as an amount of therapeutic which results in a clinically significant inhibition, amelioration or reversal of development or occurrence of a disorder or disease. The term “carrier” may refer to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water may be a preferred carrier when the pharmaceutical composition is administered intravenously. Saline solutions and aqueous dextrose and glycerol solutions may also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, may also contain wetting or emulsifying agents, or pH buffering agents. These compositions may take the form of solutions, suspensions, emulsion, tablets, pills, capsules, powders, sustained-release formulations and the like. The composition may be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. Such compositions may contain a therapeutically effective amount of the therapeutic, preferably in purified form, together with a suitable amount of carrier so as to provide the form for proper administration to the patient. The formulation may suit the mode of administration. Compositions for intravenous administration may be solutions in sterile isotonic aqueous buffer. Where necessary, the composition may also include a solubilizing agent and a local anaesthetic such as lignocaine to ease pain at the site of the injection. The ingredients may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it may be dispensed with an infusion bottle containing sterile pharmaceutical grade water or saline. Where the composition is administered by injection, an ampoule of sterile water for injection or saline may be provided so that the ingredients may be mixed prior to administration.

### EXAMPLES

Example 1. Synthesis of oligonucleotides that e.g. comprise LNA nucleotides are well known in literature. LNA monomer and oligonucleotide synthesis may be performed using the methodology referred to in Examples 1 and 2 of WO2007/11275.

Assessment of the stability of LNA oligonucleotides in human or rat plasma may be performed using the methodology referred to in Example 4 of WO2007/112754. Treatment of cultured cells with LNA-modified antisense oligonucleotides may be performed using the methodology referred to in Example 6 of WO2007/11275.

Example 2. RNA isolation and expression analysis from cultured cells and tissues is performed using the methodology referred to in Example 10 of WO2007/112754. RNAseq-based transcriptional profiling from cultured cells and tissues is performed using the methodology referred to in (Djebali et al. Nature 489: 101-108 or Chu et al. Nucleic Acid Ther. 22: 271-274 or Wang et al. Nature Reviews Genetics 10: 57-63).

### Example 3. Cell Culture

The adherent human breast adenocarcinoma cell line MCF7 (ECACC no: 86012803) was purchased from ATCC (cat. no. HTB-22™) and maintained in Eagle's Minimum Essential Medium (cat. no: M2279, Sigma Aldrich, St. Louis, MO, USA) supplied with 10% fetal bovine serum (cat. no: F4135, Sigma Aldrich, St. Louis, MO, USA), 1% non-essential amino acids (cat. no: 11140050, Thermo Fischer Scientific, Waltham, MA, USA), 1% L-glutamine (cat. no: G7513, Sigma Aldrich, St. Louis, MO, USA) and 1% penicillin/streptomycin (cat. no: P4333, Sigma Aldrich, St. Louis, MO, USA) in Nunc™ EasYFlask™ Cell Culture Flasks (cat. no: 159910, Thermo Fischer Scientific, Waltham, MA, USA). The cells were kept in in a humidified 5% CO2 incubator at 37° C. and passaged twice a week.

### Example 4. Primary Screening of the Antisense Oligonucleotide Compound Library

A library of 97 antisense oligonucleotides was designed to selectively target the first exon of adenosine kinase long isoform (ADK-L), not present in the adenosine kinase short isoform (ADK-S). The antisense oligonucleotides were synthesized by IDT (Coralville, Iowa, USA) and diluted to a stock concentration of 500 UM in nuclease-free water (cat. no: AM9938, Thermo Fischer Scientific, Waltham, MA, USA) under sterile conditions. The resuspended oligonucleotides were stored at −20° C.

The day before transfection, the MCF7 cells were seeded in 24-well Nunc™ Cell-Culture Treated Multidishes (cat. no: 142475, Thermo Fischer Scientific, Waltham, MA, USA) at 1.25×10⁵ cells/well. On the day of transfection, the cell medium was removed one hour before transfection and 475 uL of maintenance medium was added. All oligonucleotides were diluted to a final well concentration of 10 nM in Opti-MEM (cat. no: 31985-070, Thermo Fischer Scientific, Waltham, MA, USA). Lipofectamine™ RNAiMAX (cat. no: 13778150, Thermo Fischer Scientific, Waltham, MA, USA) was diluted in Opti-MEM to a final well concentration of 1.5 uL. Equal amounts of RNAiMAX and antisense oligonucleotide solutions were combined and allowed to incubate for five minutes before 25 uL of the mixture was added to the wells. As experimental controls, both a scrambled control oligonucleotide and RNAiMAX only-treated cells were used. Forty-eight hours after transfection, RNA extraction was conducted using the RNeasy mini kit (cat. no: 74106, Qiagen, Hilden, Germany) as per manufacturer's instructions. Reverse transcription was conducted using Superscript IV reverse transcriptase (cat. no: 18090010, Thermo Fischer Scientific, Waltham, MA, USA) as per manufacturer's instructions, including gDNA removal by ezDNase™ (cat. no: 11766051, Thermo Fischer Scientific, Waltham, MA, USA) and using a random hexamer primer (cat. no: SO142, Thermo Fischer Scientific, Waltham, MA, USA). The qPCR was done on a QuantStudio 6 Flex (Applied Biosystems, Waltham, MA, USA) using Taqman assays (Table 1) synthesized by Integrated DNA Technologies (Newark, NJ, USA) and TaqMan™ Universal Master Mix II, no UNG (cat. no: 4440040, Thermo Fischer Scientific, Waltham, MA, USA) as per manufacturer's instructions. All assays were designed to be exon-spanning and specificity was confirmed by blast of the primers and the efficiency of primers was tested using a five-fold dilution series. Hprt1 was used as a house-keeping gene. The ADK assay used detects only ADK-L mRNA.

TABLE 1
qPCR primers and probes

| Gene | Forward primer | Reverse primer | Probe | Cat.no: |
|---|---|---|---|---|
| ADK-L1 | GCCCAAAAAGCT | GCAGAGATGTCAAG | /56- | Custom |
| | GAAGGTGG | CAGAGGA | FAM/CGCCGCAAG/ZEN/CG | made |
| | | | CTGAGAGAA/3IABKFQ/ | |
| ADK-L2 | TGGGCTGTAGAG | AGCAGAGATGTCAA | /56- | Custom |
| | CCAAAGTG | GCAGAGG | FAM/GGAGCGCGA/ZEN/AG | made |
| | | | ATGGCAGCT/3IABKFQ/ | |
| Hprt1 | GCGATGTCAATA | TTGTTGTAGGATATG | /56- | Hs.PT.58v. |
| | GGACTCCAG | CCCTTGA | FAM/AGCCTAAGA/ZEN/TGA | 45621572 |
| | | | GAGTTCAAGTTGAGTTTGG/ | |
| | | | 3IABKFQ/ | |

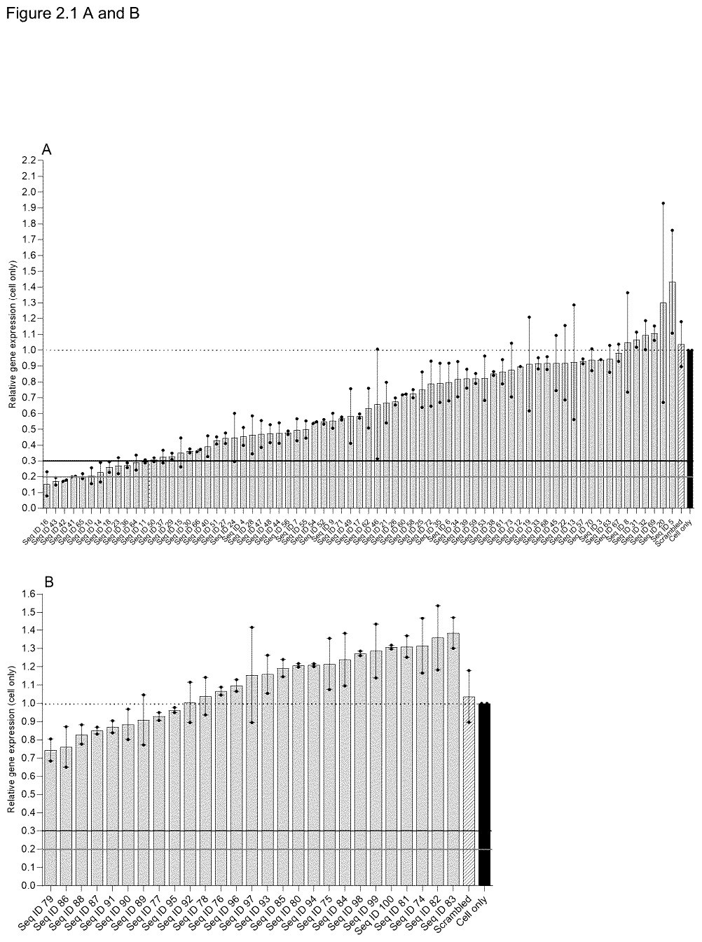

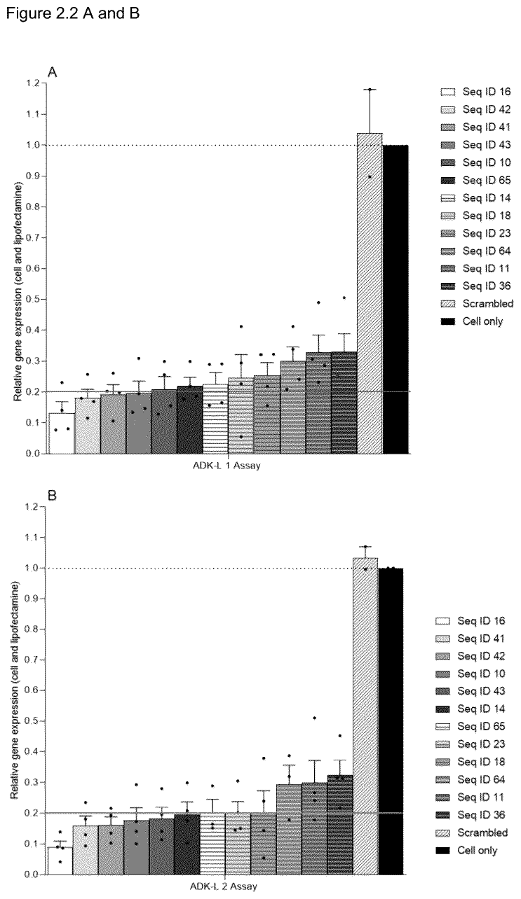

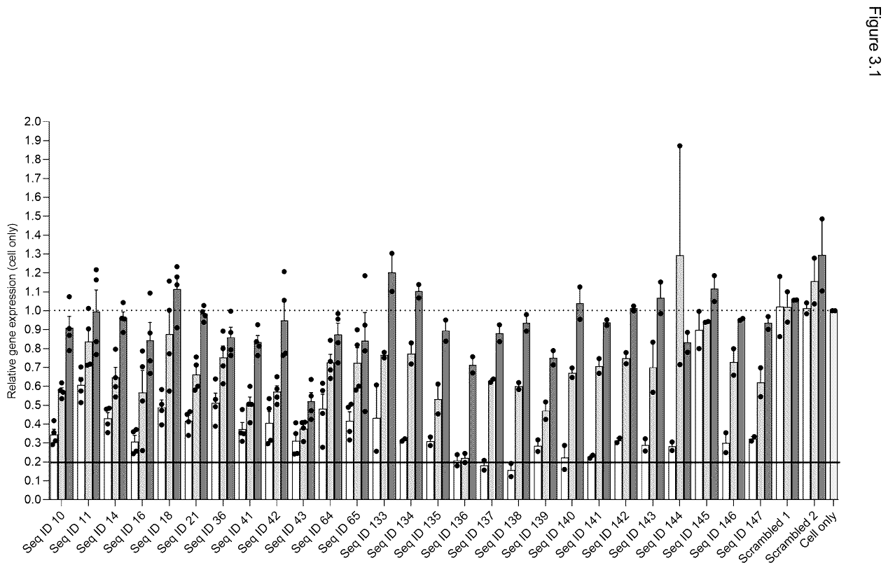

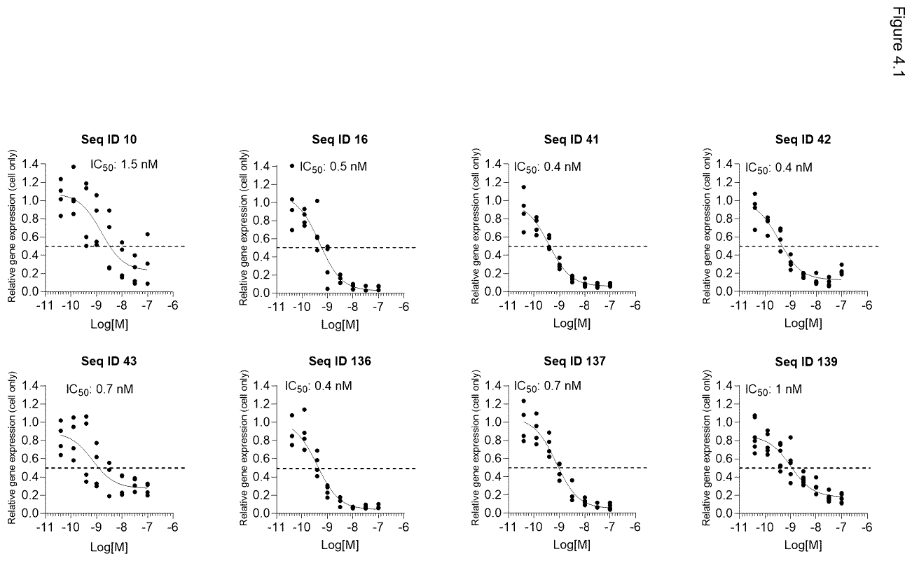

All data were calculated in Microsoft Excel and visualized in Prism ver. 9.1.1, (GraphPad, San Diego, CA, USA). qPCR results were analysed using the ΔΔCt method using cells treated with RNAiMAX only as reference. The first screening (FIG. 2.1) was done with n=1 with two technical replicates and is depicted as mean±SEM. The top twelve candidates were chosen (SEQ ID NO's: 16, 43, 42, 41, 65, 10, 14, 18, 23, 36, 64, and 11, and the level of ADK-L mRNA knockdown was confirmed in a follow-up experiment (n,N=2,3-4, (n=biological replicate, N=technical replicate)) depicted as mean±SEM in FIG. 2.2. To further validate the results, RNA from cells treated with the top antisense oligonucleotide candidates were analysed with a second qPCR assay specific for ADK-L.

### Example 5: Knockdown of ADK-L by Selected Antisense Oligonucleotides

The transfections and the qPCR (ADK-L 1 assay only) were done as in example 2 except that the antisense oligonucleotide concentrations were either 5, 1 or 0.2 nM. The experiment was repeated giving two biological replicates with two technical replicates each. FIG. 3.1 shows the results of the dose-response study, based on which compounds SEQ ID NO's: 10, 16, 41, 42, 43, 136, 137, and 139 were selected for IC50 value testing.

### Example 6: Determination of IC50 Values for Selected ADK-L Antisense Oligonucleotides in Cultured Cell Lines

The transfections and the qPCR were done as in example 3, except that the cells were transfected with a range of antisense oligonucleotides concentrations in 3-fold dilutions from 90 nM to 0.004 nM. The relative level of ADK-L as determined by qPCR was plotted against log(M) in Graphpad Prism (version 9.0.2, GraphPad Software). The dose-response curves were fitted using 3-parameter non-linear fit and IC₅₀ values calculated in nM. The experiment was repeated giving two to three biological replicates with two technical replicates each.

FIG. 4.1 shows the dose-response curves and the IC₅₀ values of ADK-L antisense oligonucleotides.

### Example 7: RNA-Sequencing in Cultured Cell Lines

The transfection of cells was done as in above experiments with the exception that antisense oligonucleotide concentrations were 3 and 30 nM, respectively. The experiment was repeated giving three biological replicates. RNA was isolated from cell pellets using miRNeasy Mini Kit (cat. no: 217004, Qiagen), contaminant genomic DNA was removed by using the RNase-free DNase set (cat. no: 79254, Qiagen). The final RNA quality was evaluated using an RNA Nano chip on the Bioanalyzer 2100 (cat. no: 5067-1511, Agilent technologies, Santa Clara, CA, USA).

Isolated RNA samples were rRNA depleted and prepared for sequencing using SMARTer Stranded Total RNA Sample Prep Kit—HI Mammalian (cat. no: 38229000, Takara Bio Europa). The rRNA depletion was performed using RiboGone and the remaining RNA was purified using AMPure XP beads (cat no. A63881, Beckman Coulter, Brea, CA, USA) and library construction was done according to the manufacturer's protocol. The final libraries were size-selected (150-500 bp) on a Pippin Prep (Sage Science, Inc. Beverly, MA, USA), quality controlled on the Bioanalyzer 2100 using the Qubit and high sensitivity chip (Agilent) and quantified using the KAPA library quantification kit (Kapa Biosystems, Wilmington, MA, USA). RNA-seq was performed on the Novaseq 6000 S4 at Novogene (Cambridge, UK).

Sequencing data were pre-processed by removing adapter sequence and trimming away low-quality bases with a Phred score below 20 using Trim Galore (v0.4.1). Quality control was performed using FastQC and MultiQC¹ to ensure high quality data. ¹ Philip Ewels and others, ‘MultiQC: Summarize Analysis Results for Multiple Tools and Samples in a Single Report’, Bioinformatics, 32.19 (2016), 3047-48<https://doi.org/10.1093/bioinformatics/btw354>.

Quantification of gene expression was performed by mapping the filtered reads to the human genome (hg19) using STAR². The software FeatureCounts was used to quantify the number of reads mapping to each gene using gene annotation from Gencode V37³. ² Alexander Dobin and others, ‘STAR: Ultrafast Universal RNA-Seq Aligner’, Bioinformatics, 29.1 (2013), 15-21 <https://doi.org/10.1093/bioinformatics/bts635>.³ Yang Liao, Gordon K Smyth, and Wei Shi, ‘FeatureCounts: An Efficient General Purpose Program for Assigning Sequence Reads to Genomic Features’, Bioinformatics, 30.7 (2014), 923-30 <https://doi.org/10.1093/bioinformatics/btt656>.

Differential expression analysis was performed using DESeq2 in R on gene expression levels⁴. Predicted gene targets for were found for each antisense oligonucleotide by in silico analysis using GGGenome as referenced⁵. The sequence of each antisense oligonucleotide was matched against both mature spliced mRNA sequences (splice) and against unspliced pre-mRNA sequences (presplice) from RefSeq allowing up to a total of three insertions, deletions, or mismatches. The sum of insertions, deletions, and mismatches for each antisense oligonucleotide match were denoted as the “distance” (d) representing the quality of the predicted target site; d=0 means a perfect match and d=3 means three insertions, deletions, or mismatches in the binding between antisense oligonucleotide and (pre-)mRNA. Predicted mRNA and pre-mRNA antisense oligonucleotide targeting was compared to gene expression and differential expression analysis from RNA-seq to estimate which genes are differentially expressed due to antisense oligonucleotide off-targeting. All plotting was done in R. ⁴ Michael I Love, Wolfgang Huber, and Simon Anders, ‘Moderated Estimation of Fold Change and Dispersion for RNA-Seq Data with DESeq2’, Genome Biology, 15.12 (2014), 550<https://doi.org/10.1186/s13059-014-0550-8>.⁵ Tokuyuki Yoshida and others, ‘Evaluation of Off-Target Effects of Gapmer Antisense Oligonucleotides Using Human Cells’, Genes to Cells, 24.12 (2019), 827-35<https://doi.org/https://doi.org/10.1111/gtc.12730>.

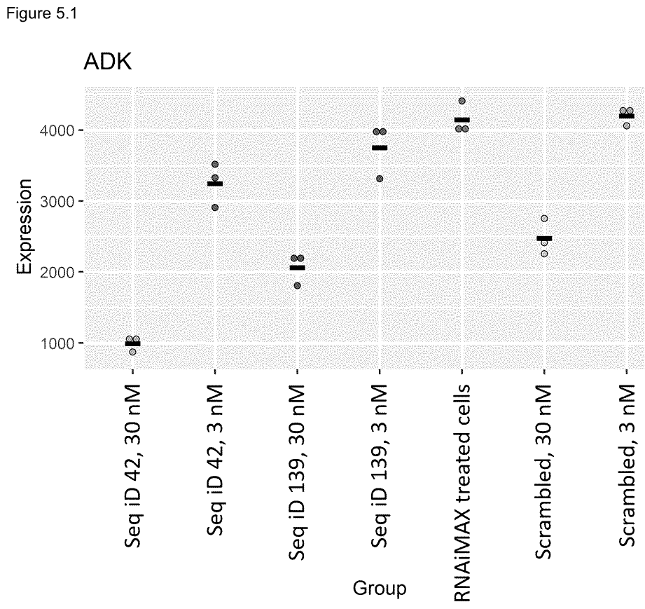

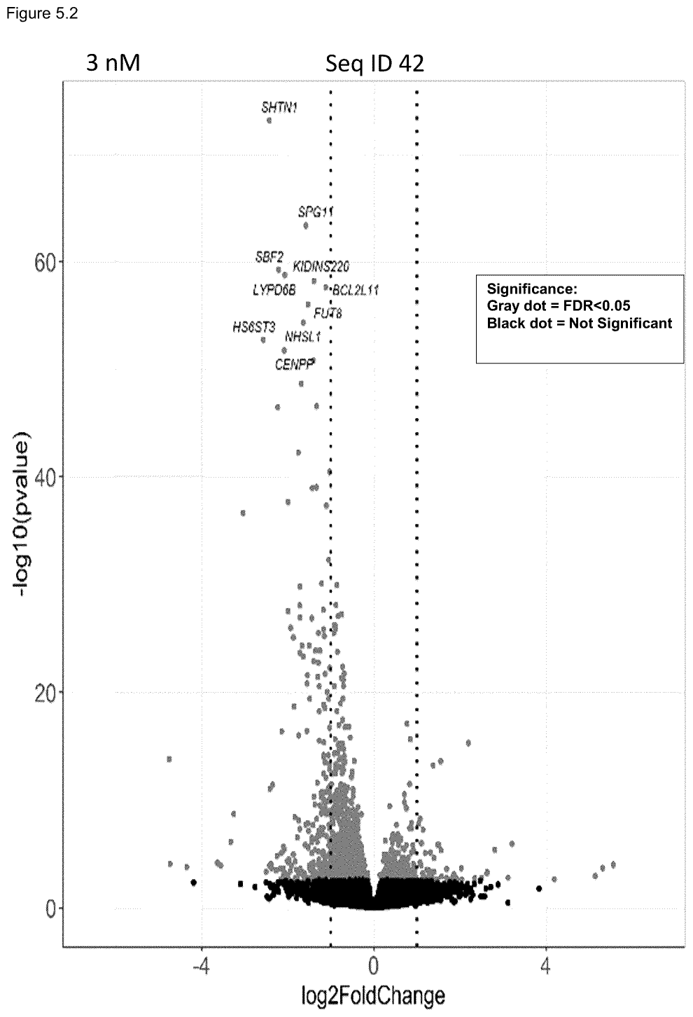

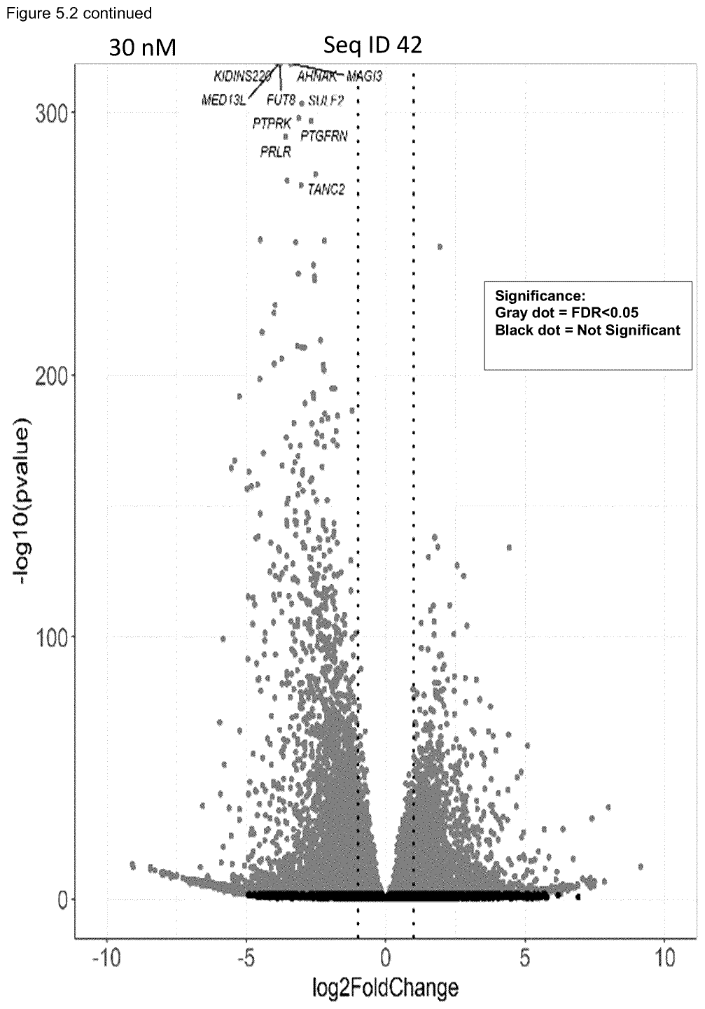

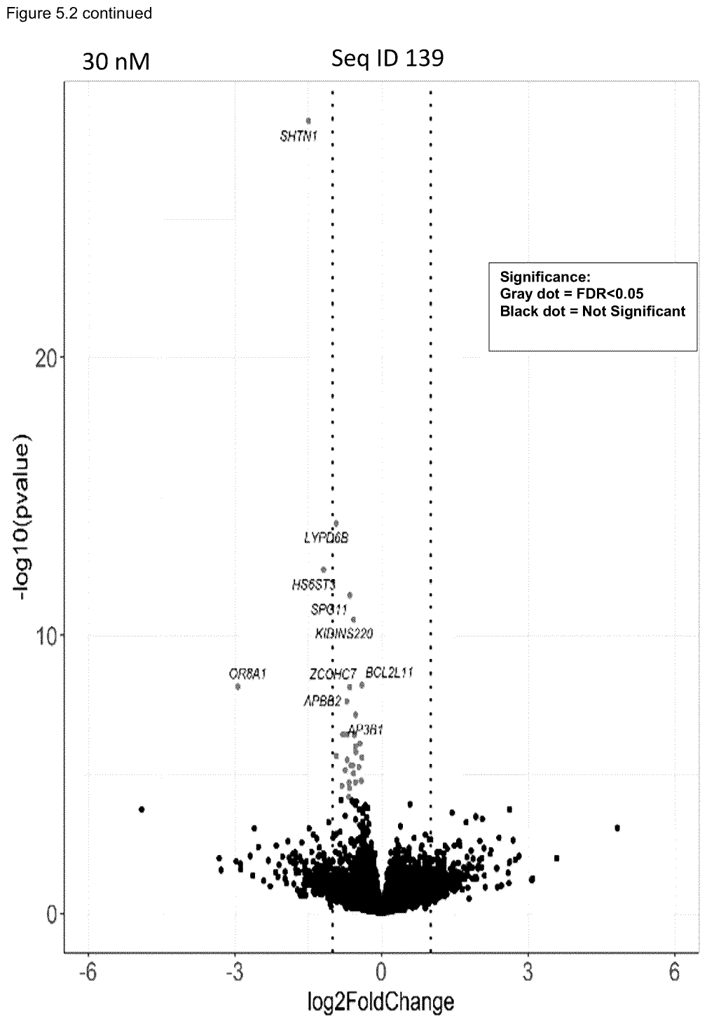

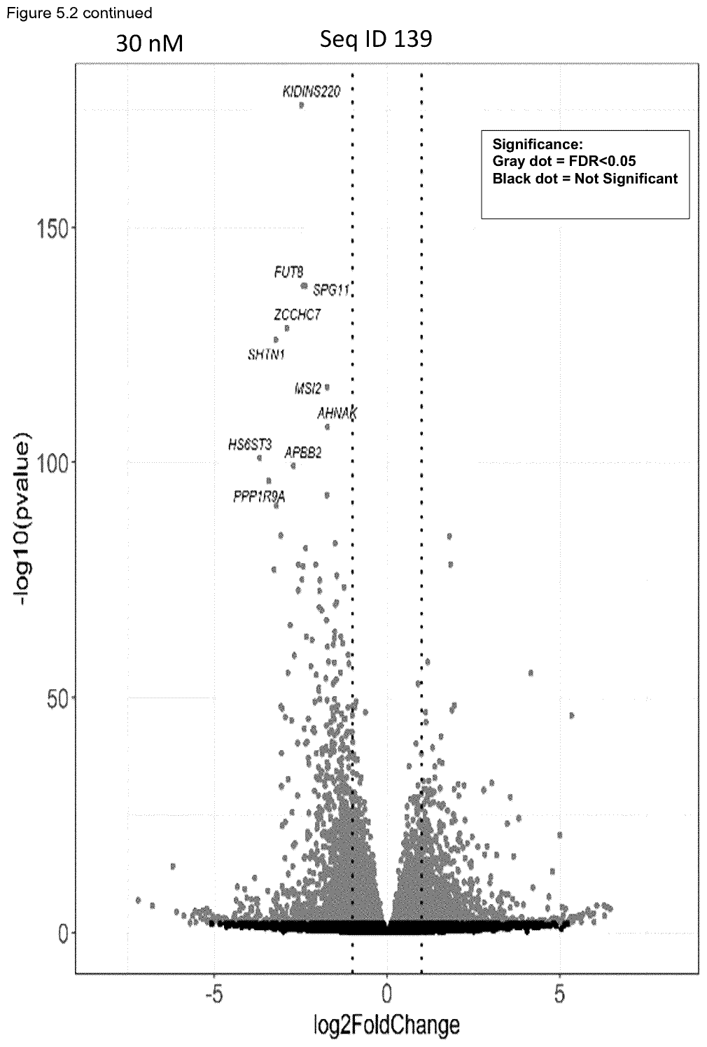

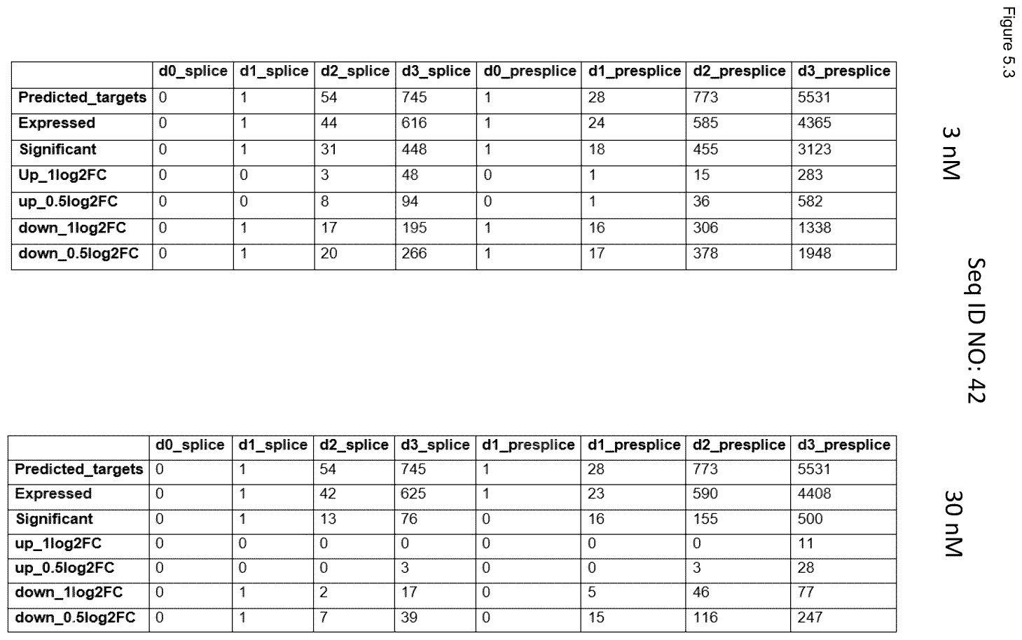

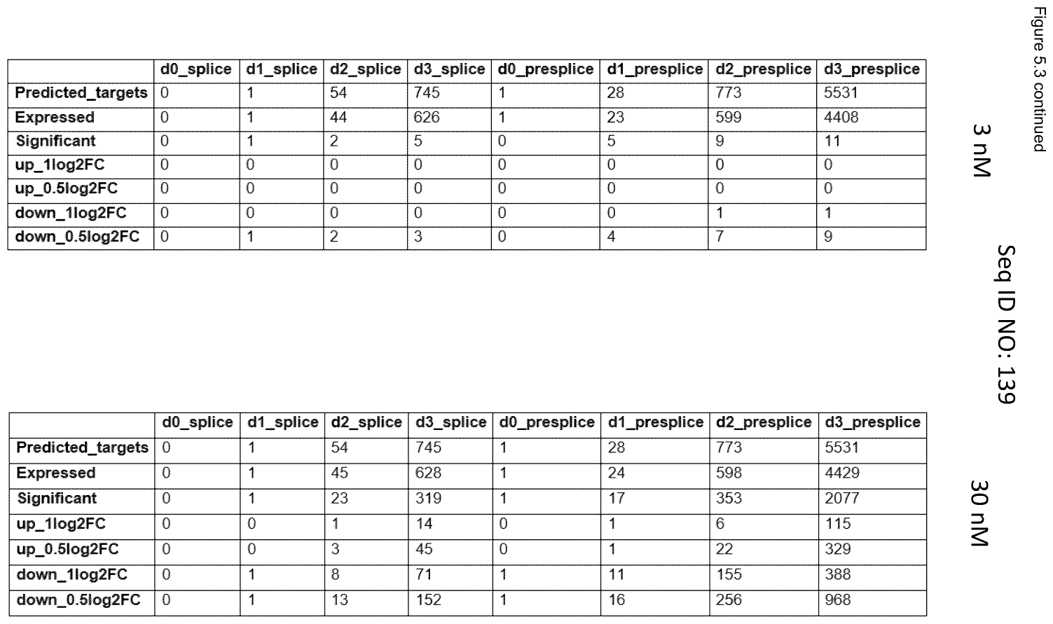

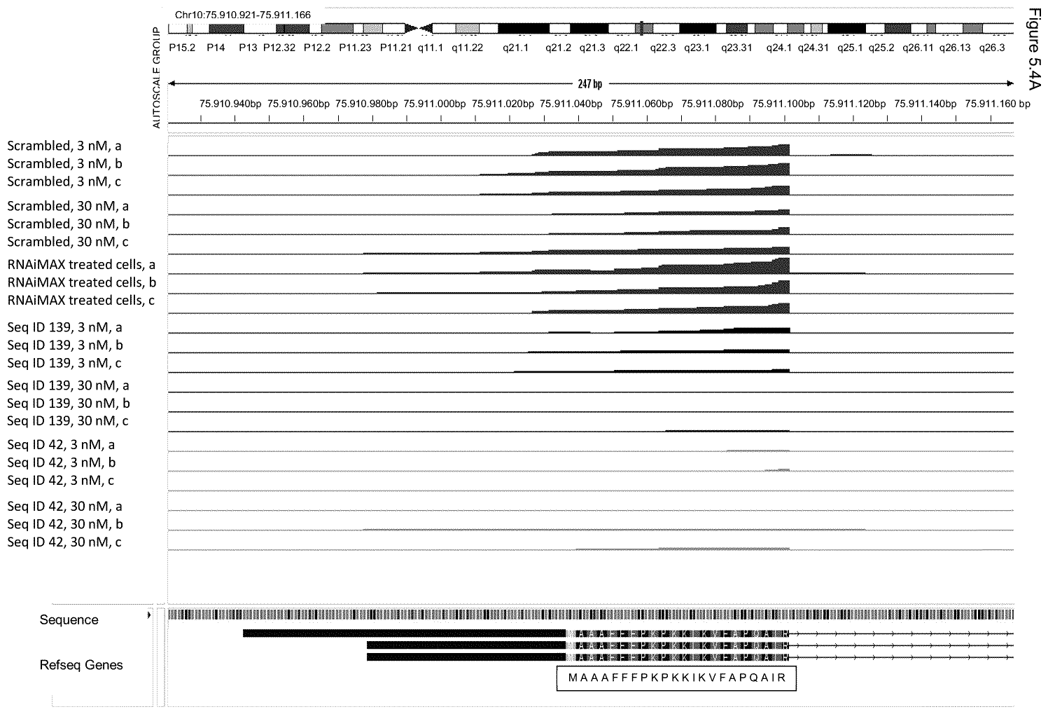

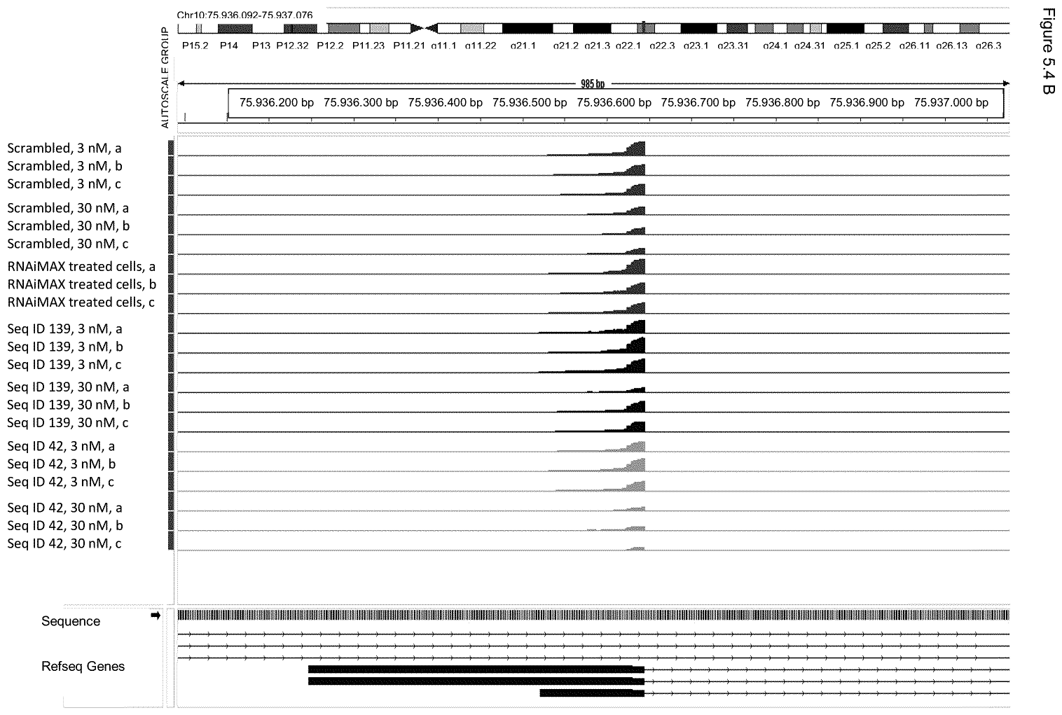

To evaluate the effect of antisense oligonucleotide treatment on the ADK expression, the expression level was normalised and compared across samples (FIG. 5.1).

FIG. 5.1 shows normalized mRNA expression values for ADK (both isoforms), n=3.

To evaluate the effects of the ADK-L specific antisense oligonucleotides on the whole transcriptome, differential gene expression analysis was performed, and the resultant data visualized in volcano plots (FIG. 5.2): differential gene expression analysis of cells treated with Seq ID 42 (left) and Seq ID 139 (right). The volcano plots show the different levels of transcripts between Seq ID 42, Seq ID 139 and mock treated cells, correlating the changes in RNA expression between antisense oligonucleotide-treated and mock-treated groups with the significance of the differential expression. The x-axis denoted relative change in expression while the y-axis denotes the significance. Each dot denotes a specific RNA. Black dots represent non-significant changes, while gray dots display significant values.

To examine whether a change in RNA expression could be ascribed to either 1) a direct effect by targeting other sequences in the transcriptome or 2) a downstream secondary consequence of the direct effects an initial in silico analysis was performed, using the antisense oligonucleotide sequences to predict all potential target sites within the 1) spliced transcriptome (cytoplasmic) and the 2) unspliced transcriptome (nuclear). This was done for either target sites with 0, 1, 2 or 3 insertions, deletions, or mismatches, collectively called the distance (d). A distance of 0 was only observed for antisense oligonucleotide binding to ADK RNA. The results are depicted in FIG. 5.3.

To evaluate the ADK-L specific effects of SEQ ID NO: 42 and SEQ ID NO: 139, further analysis was done on the ADK-L isoform alone. The ADK-L and —S isoforms vary by the transcriptional start sites with the two isoforms using different first exons. The ADK-L isoform includes an upstream start exon and skips the ADK-S first exon. This transcriptional difference was used to discriminate between the two isoforms by manual investigation of the specific first exons in the ADK-L and ADK-S isoforms showing the ADK-L specific knockdown by Seq ID 42 and 139 at 3 nM but not at 30 nM where SEQ ID NO: 42 influenced the ADK-S isoform as well (FIG. 5.4).

FIG. 5.4 shows a close-up of ADK-L to ADK-S first exons. IGV genome browser views showing read mapping on first exons of ADK-L (A) and ADK-S(B).

### Embodiments

1 An antisense oligonucleotide complementary to ADK mRNA (SEQ ID NO: 1) comprising a sequence of 10-30 nucleotides in length, wherein the antisense oligonucleotide has at least one affinity-enhancing nucleotide analogue and wherein said antisense oligonucleotide comprises at least one phosphorothioate internucleoside linkage.
2 The antisense oligonucleotides according to embodiment 1), wherein the antisense oligonucleotide is specific for ADK-L and is complementary to any of SEQ ID NO: 2, 120, 121, or 122-132.
3 The antisense oligonucleotide according to embodiment 1) or 2), which comprises a motif according to any of SEQ ID NO: 101-119.
4 The antisense oligonucleotide according to any of embodiments 1)-3), wherein the antisense oligonucleotide contains a contiguous stretch of at least five, such as at least six or at least 7 contiguous DNA nucleotides.
5 The antisense oligonucleotide according to any of embodiments 1)-3), wherein the antisense oligonucleotide does not contain a contiguous stretch of more than 4 DNA nucleotides, such as not more than 3 contiguous DNA nucleotides.
6 The antisense oligonucleotide according to any of embodiments 1)-5), wherein the antisense oligonucleotide comprises a sequence of 14-19 nucleotides in length.
7 The antisense oligonucleotide according to any of embodiments 1)-6), wherein the affinity-enhancing nucleotide analogue is selected from the list of LNA, tricyclo-DNA, 2′-Fluoro, 2′-O-methyl, 2′methoxyethyl (2′MOE), 2′ cyclic ethyl (cET), UNA, 2′fluoro and Conformationally Restricted Nucleoside (CRN).
8 The antisense oligonucleotide according to any of embodiments 1)-7), wherein the antisense oligonucleotide, comprises at least one LNA.
9 The antisense oligonucleotide according to any of embodiments 1)-8), wherein the antisense oligonucleotide comprises from 30-55% LNA.
10 The antisense oligonucleotide according to embodiment 9), wherein the antisense oligonucleotide further comprises one or more nucleosides that are anyone of tricyclo-DNA, 2′-Fluoro, 2′-O-methyl, 2′methoxyethyl (2′MOE), 2′ cyclic ethyl (CET), UNA, 2′fluoro and Conformationally Restricted Nucleoside (CRN).
11 The antisense oligonucleotide according to any of embodiments 1)-10), wherein the LNA is Beta-D-Oxy LNA.
12 The antisense oligonucleotide according to any of embodiments 1)-11), wherein all the internucleoside bonds are phosphorothioate bonds.
13 The antisense oligonucleotide according to any of embodiments 1)-12), wherein the antisense oligonucleotide is anyone of SEQ ID NO's 3-73 or SEQ ID NO's 133-147, such as anyone of SEQ ID NO's: 10, 11, 14, 16, 18, 23, 36, 41, 42, 43, 64, 65, 133, 134, 135, 136, 137, 138, 139, 140, 141, 142, 143, 144, 145, 146, or 147; such as anyone of SEQ ID NO's: 10, 16, 41, 42, 43, 136, 137, or 139; such as anyone of:

(SEQ ID NO 10)
5′ CTttatacttATtAGgAAG 3′
or
(SEQ ID NO 16)
5′ CTttatactTaTTaggAAG 3′
or
(SEQ ID NO 41)
5′ AGctTtTtAAagcaaCAG 3′
or
(SEQ ID NO 42)
5′ CTTtgggattTCaGAAA 3′
or
(SEQ ID NO 43)
5′ CTttgggattTCaGAAA 3′
or
(SEQ ID NO 136)
5′ CTttatactTaTTaGgAAG 3′
or
(SEQ ID NO 137)
5′ CTttatacttATtaGgAAG 3′
or
(SEQ ID NO 139)
5′ CTttgggatTTCaGAAA 3′

and wherein upper case letters denote LNA, lower case letters are DNA, upper case C is LNA 5-methylcytosine, LNA is beta-D-oxy LNA and all internucleoside bonds are phosphorothioate linkages.
14 The antisense oligonucleotide according to any of embodiments 1)-12), wherein the antisense oligonucleotide is anyone of SEQ ID NO's 74-100.
15 A composition comprising the antisense oligonucleotides according to anyone of embodiments 1)-14).
16 The antisense oligonucleotide according to any of embodiments 1)-14) or the composition according to embodiment 15), for use as a medicament.
17 The antisense oligonucleotide according to any of embodiments 1)-14), or the composition according to embodiment 15), wherein said antisense oligonucleotide or composition is for use as a medicament, preferably wherein said medicament is for use in the preventive, curative or disease modifying treatment, alleviation, pre-emptive treatment or prophylaxis of a disease wherein modification of ADK activity is beneficial.
18 The antisense oligonucleotide or composition according to any of embodiments 1)-15), wherein the use is for the preventive, curative or disease modifying treatment, alleviation, amelioration, pre-emptive treatment or prophylaxis of CNS or PNS disease.
19 The antisense oligonucleotide or composition according to any of embodiments 1)-15), wherein said antisense oligonucleotide or said composition is for use in the preventive, curative or disease modifying treatment, alleviation, pre-emptive treatment or prophylaxis of a disease of the CNS or PNS, such as a psychiatric, neurological disorder, a neurodegenerative disorder or a neurodevelopmental disorder.
20 The antisense oligonucleotide or composition according to any of embodiments 1)-15), for use as a neuroprotective agent.
21 The antisense oligonucleotide or composition according to any of embodiments 1)-15), for use in the preventive, curative or disease modifying treatment, alleviation, pre-emptive treatment or prophylaxis of a disease of the CNS or PNS, a psychiatric, a neurological disorders, a neurodegenerative disorders, a neurodevelopmental disorders, a central and peripheral nervous system diseases associated with cellular trauma and inflammation, neuronal damage, hippocampal damage, traumatic brain injury, a memory disorder, hippocampal sclerosis, Parkinsons Disease, multiple sclerosis, acute spinal cord injury, amyotrophic lateral sclerosis, ataxia, bell's palsy, Charcot-Marie-Tooth, a headache, Horton's headache, migraine, pick's disease, progressive supranuclear palsy, multi-system degeneration, a motor neuron disease, Huntington's disease, prion disease, Creutzfeldt-Jakob disease, corticobasal degeneration, primary progressive aphasia or symptoms or effects thereof.
22 The antisense oligonucleotide or composition according to any of embodiments 1)-15), for use in the preventive, curative or disease modifying treatment of epilepsy.
23 The antisense oligonucleotide or composition according to any of embodiments 1)-15), for use in the preventive, curative or disease modifying treatment of seizures.
24 The antisense oligonucleotide or composition according to any of embodiments 1)-15), wherein said antisense oligonucleotide or said composition is for use in the preventive, curative or disease modifying treatment, alleviation, pre-emptive treatment or prophylaxis of epilepsy and/or seizures, preferably a treatment resistant epilepsy, acquired, genetic and/or idiopathic epilepsy, therapy resistant epileptic syndromes, drug resistant epilepsy, pharmacy resistant focal epilepsy, spontaneous seizures, therapy resistant seizures, focal epilepsy, generalised epilepsy or status epilepticus.
25 The antisense oligonucleotide or composition according to any of embodiments 1)-15), for use in the preventive, curative or disease modifying treatment, alleviation, pre-emptive treatment or prophylaxis of epilepsy, drug resistant epilepsy, pharmacoresistant focal epilepsy, seizures, spontaneous seizures, therapy resistant seizures, focal epilepsy, preferably wherein said focal epilepsy is focused in the frontal lobe, the parietal lobe, the occipital lobe or the temporal lobe, generalised epilepsy, preferably wherein said generalised epilepsy is selected among absences, myoclonic seizures, tonic-clonic seizures, tonic seizures, atonic seizures, clonic seizures and spasms, status epilepticus, epileptogenesis induced by acute brain injury, autosomal dominant nocturnal frontal lobe epilepsy, continuous spike-and-waves during slow sleep, dravet syndrome, epilepsy developed after apoplexy, epileptic encephalopathy, gelastic epilepsy, absences, benign neonatal seizures, Jeavons syndrome, Juvenile myoclonic epilepsy, Landau-Kleffner Syndrom, Lennox-Gastaut syndrome, Mesial temporal lobe epilepsy, myoclonic astatic epilepsy, Ohtahara Syndrom, Panayiotopoulos syndrome, PCDH19 syndrom, benign childhood epilepsy with centrotemporal spikes, Sturge-Weber syndrome, symptomatic focal epilepsy, transient epileptic amnesia and West syndrome, and/or glioma-associated epilepsy.
26 The antisense oligonucleotide or composition according to any of embodiments 1)-15), for use in the preventive, curative or disease modifying treatment, alleviation, pre-emptive treatment or prophylaxis of pain, preferably wherein said pain is a chronic pain, a neuropathic pain, a chemotherapy-induced neuropathic pain, a migraine, a headaches, hyperalgesia, allodynia and/or fibromyalgia.
27 The antisense oligonucleotide or composition according to any of embodiments 1)-15), for use in the preventive, curative or disease modifying treatment of pain.
28 The antisense oligonucleotide or composition according to any of embodiments 1)-15), for use in the preventive, curative or disease modifying treatment, alleviation, pre-emptive treatment or prophylaxis of pain, chronic plain, neuropathic pain, chemotherapy-induced neuropathic pain, migraine, including migraine with aura and migraine without aura, a primary headache, a tension headache, a cluster headache, Hortons headache, a chronic daily headache, a sinus headache, a posttraumatic headache, an exercise headache, hemicrannia continua, hypnic headache, hyperalgesia, thermal hyperalgesia, allodynia, tactile allodynia and/or fibromyalgia.
29 The antisense oligonucleotide or composition according to any of embodiments 1)-15), for use in the preventive, curative or disease modifying treatment, alleviation, pre-emptive treatment or prophylaxis of a psychiatric disorder, a cognitive disorder, a sleep disorder, a cardiovascular disorder, a respiratory disorder, a cancer, a renal disorder, an inflammation or a metabolic disorder.
30 The antisense oligonucleotide or composition according to any of embodiments 1)-15), for use in the preventive, curative or disease modifying treatment, alleviation, pre-emptive treatment or prophylaxis of a psychiatric disorder, a neuropsychiatric disorder, anxiety, depression, bipolar disorder, attention deficit hyperactive disorder, attention deficit disorder, autism, Asperger's, Tourette, schizophrenia, paranoid schizophrenia, hebephrenic schizophrenia, catatonic schizophrenia, undifferentiated schizophrenia, residual schizophrenia, simple schizophrenia or unspecified schizophrenia.
31 The antisense oligonucleotide or composition according to any of embodiments 1)-15), for use in the preventive, curative or disease modifying treatment, alleviation, pre-emptive treatment or prophylaxis of a cognitive disorder, cognitive impairment, dementia, Alzheimer disease, vascular dementia, frontotemporal dementia or Lewy bodies dementia.
32 The antisense oligonucleotide or composition according to any of embodiments 1)-15), for use in the preventive, curative or disease modifying treatment, alleviation, pre-emptive treatment or prophylaxis of a sleep disorders.
33 The antisense oligonucleotide or composition according to any of embodiments 1)-15), for use as a sleep modulating agent.
34 The antisense oligonucleotide or composition according to any of embodiments 1)-15), for use in sleep promotion.
35 The antisense oligonucleotide or composition according to any of embodiments 1)-15), for use in the preventive, curative or disease modifying treatment, alleviation, pre-emptive treatment or prophylaxis of a cardiovascular disorders, a peripheral artery disease, postoperative atrial fibrillation, heart failure, chronic heart failure, intracerebral haemorrhage-induced brain injury, stroke, cerebral ischemia or ischaemia.
36 The antisense oligonucleotide or composition according to any of embodiments 1)-15), for use in the preventive, curative or disease modifying treatment, alleviation, pre-emptive treatment or prophylaxis of a respiratory disorder, asthma or chronic obstructive pulmonary disease.
37 The antisense oligonucleotide or composition according to any of embodiments 1)-15), for use in the preventive, curative or disease modifying treatment, alleviation, pre-emptive treatment or prophylaxis of a cancer, a cancer in the nerve system, glioma, glioblastoma, hepatic cancer or a cancer metastasis.
38 The antisense oligonucleotide or composition according to any of embodiments 1)-15), for use in the preventive, curative or disease modifying treatment, alleviation, pre-emptive treatment or prophylaxis of a renal disorder, renal injury, renal inflammation, albuminuria or glomerular injury.
39 The antisense oligonucleotide or composition according to any of embodiments 1)-15), for use in the preventive, curative or disease modifying treatment, alleviation, pre-emptive treatment or prophylaxis of inflammation.
40 The antisense oligonucleotide or composition according to any of embodiments 1)-15), for use in the preventive, curative or disease modifying treatment, alleviation, pre-emptive treatment or prophylaxis of an inflammatory disorder, oxidative stress, inflammation, apoptosis, arthritis, osteoarthritis, rheumatoid arthritis, and the pain associated with these conditions, encephalitis, meningitis, human Rasmussen encephalitis, inflammation of cerebral cortex and/or hippocampus, progressive cognitive deterioration, colitis, ulcerative colitis or inflammatory bowel disease.
41 The antisense oligonucleotide or composition according to any of embodiments 1)-15), for use in the treatment, alleviation, pre-emptive preventive, curative or disease modifying treatment or prophylaxis of a metabolic disorder, preferably diabetes, more preferably type 1 or type 2 diabetes.
42 The antisense oligonucleotide or composition according to any of embodiments 1)-15), for use in the preventive, curative or disease modifying treatment, alleviation, pre-emptive treatment or prophylaxis of Prader-Willis Syndrome, Anglemans Syndrome, neurofibromatosis, an angiogenesis related disease, promotion of angiogenesis, a disorder of the retina, preferably diabetic retinopathy or hearing loss.
43 The antisense oligonucleotide or composition according to any of the preceding embodiments, wherein said antisense oligonucleotide or composition is administered by systemic administration, subcutaneous administration, nasal, intrathecal administration, intraventricular administration into the CNS or intravenous administration.
44 The antisense oligonucleotide or composition according to any of the preceding embodiments, for use in combination with one or more other active pharmaceutical ingredients for the preventive, curative or disease modifying treatment of any of the diseases of embodiments 16)-42).
45 The use according to embodiment 44), wherein the other active pharmaceutical ingredient is an ingredient made for preventive, curative or disease modifying treatment of the diseases of any of embodiments 16)-42).
46 The use according to embodiment 44) or 45), wherein the other pharmaceutical ingredient is an antisense oligonucleotide targeting miR-27b or miR-134.
47 A pharmaceutical composition comprising an effective dosage of the antisense oligonucleotide according to anyone of the preceding embodiments and a pharmaceutically acceptable carrier.
48 A pharmaceutical composition comprising an effective dosage of the antisense oligonucleotide according to anyone of the preceding embodiments, wherein said antisense oligonucleotide is the sole active pharmaceutical ingredient.
49 The pharmaceutical composition according to embodiment 47) or 48), wherein the composition is for use according to any of embodiments 16)-46).
50 The pharmaceutical composition according to any of embodiments 47)-49), wherein the composition is for intratecal administration, or for intracerebroventricular administration.
51 The pharmaceutical composition according to embodiment 50), wherein said composition is administered in a pump, preferably wherein said pump is a mini pump, more preferably wherein said mini pump is a mini-osmotic pump.
52 The pharmaceutical composition according to any of embodiments 50)-51), wherein said composition is for intraventricular administration facilitated by an intraventricular catheter, preferably wherein said catheter is attached to a reservoir, preferably wherein said reservoir is an Ommaya reservoir.
53 The pharmaceutical composition according to any of embodiments 47)-52), wherein said composition is administered with an interval of anyone of 1 day, 2 days, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 days.
54 The pharmaceutical composition according to any of embodiments 47)-52), wherein said composition is administrated with an interval of between 1-200 days, 10-190 days, 20-180 days, 30-170 days, 40-160 days, 50-150 days, 60-140 days, 70-130 days, 80-120 days, 90-110 days or preferably about 100 days.
55 The antisense oligonucleotide or the composition according to any of embodiments 1)-15), for use in a method of treating the diseases according to any of embodiments 16)-42).
56 A method of treatment of the diseases according to any of embodiments 16)-42), by use of the antisense oligonucleotides according to any of embodiments 1)-14) or the composition according to embodiment 15) or the pharmaceutical composition according to any of embodiments 47)-54).
57 The use according to any of embodiments 16)-46), or method according to embodiment 56), wherein the treatment is anyone of preventive, curative or disease modifying.
58 A method of diagnosing a disease according to any of embodiments 16)-42) by use of the antisense oligonucleotide according to any of embodiments 1)-14) or the composition according to embodiment 15).

1. An antisense oligonucleotide complementary to Adenosine kinase (ADK) mRNA (SEQ ID NO: 1) comprising a sequence of 10-30 nucleotides in length, wherein the antisense oligonucleotide has at least one affinity-enhancing nucleotide analogue and, wherein said antisense oligonucleotide comprises at least one phosphorothioate internucleoside linkage.

2-16. (canceled)

17. The antisense oligonucleotides according to claim 1, wherein the antisense oligonucleotide is specific for ADK-L and is complementary to any of SEQ ID NO: 2, 120, 121, or 122-132.

18. The antisense oligonucleotide according to claim 1, wherein the antisense oligonucleotide contains a contiguous stretch of at least five contiguous DNA nucleotides.

19. The antisense oligonucleotide according to claim 1, wherein the antisense oligonucleotide does not contain a contiguous stretch of more than 4 DNA nucleotides.

20. The antisense oligonucleotide according to claim 1, wherein the antisense oligonucleotide comprises a sequence of 14-19 nucleotides in length.

21. The antisense oligonucleotide according to claim 1, wherein the affinity-enhancing nucleotide analogue is selected from a locked nucleic acid (LNA), tricyclo-DNA, 2′-Fluoro, 2′-O-methyl, 2′methoxyethyl (2′MOE), 2′ cyclic ethyl (CET), an unlocked nucleic acid (UNA), 2′fluoro, or Conformationally Restricted Nucleoside (CRN).

22. The antisense oligonucleotide according to claim 1, wherein the antisense oligonucleotide, comprises at least one LNA.

23. The antisense oligonucleotide according to claim 1, wherein the antisense oligonucleotide is anyone of SEQ ID NO's 3-73, or anyone of SEQ ID NO's 74-100, or anyone of SEQ ID NO's 133-147.

24. The antisense oligonucleotide according to claim 1, wherein the antisense oligonucleotide is anyone of SEQ ID NO's: 10, 11, 14, 16, 18, 23, 36, 41, 42, 43, 64, 65, 133, 134, 135, 136, 137, 138, 139, 140, 141, 142, 143, 144, 145, 146, or 147.

25. The antisense oligonucleotide according to claim 1, wherein the antisense oligonucleotide is anyone of SEQ ID NO's:
(SEQ ID NO 10)
5′ CTttatacttATtAGgAAG 3′
or
(SEQ ID NO 16)
5′ CTttatactTaTTaggAAG 3′
or
(SEQ ID NO 41)
5′ AGctTtTtAAagcaaCAG 3′
or
(SEQ ID NO 42)
5′ CTTtgggattTCaGAAA 3′
or
(SEQ ID NO 43)
5′ CTttgggattTCaGAAA 3′
or
(SEQ ID NO 136)
5′ CTttatactTaTTaGgAAG 3′
or
(SEQ ID NO 137)
5′ CTttatacttATtaGgAAG 3′
or
(SEQ ID NO 139)
5′ CTttgggatTTCaGAAA 3′
and wherein upper case letters denote LNA, lower case letters are DNA, upper case C is LNA 5-methylcytosine, LNA is beta-D-oxy LNA and all internucleoside bonds are phosphorothioate linkages.

26. The antisense oligonucleotide according to claim 1, wherein the oligonucleotide is linked to a delivery vehicle or formulated for targeted delivery.

27. A pharmaceutical composition comprising the antisense oligonucleotide of claim 1 and a pharmaceutically acceptable carrier.

28. A method for treating, alleviating, pre-emptive treatment or prophylaxis of a disease of the central nervous system (CNS) or peripheral nervous system (PN)S that relies on expression of ADK-L, said method comprising administering an effective dosage of an antisense oligonucleotide complementary to ADK mRNA (SEQ ID NO: 1) comprising a sequence of 10-30 nucleotides in length, wherein the antisense oligonucleotide has at least one affinity-enhancing nucleotide analogue and, wherein said antisense oligonucleotide comprises at least one phosphorothioate internucleoside linkage or a pharmaceutical composition comprising said antisense oligonucleotide to a subject in the need thereof.

29. The method of claim 28, wherein the disease of the CNS or PNS is a neurological disorder.

30. The method of claim 28, wherein the neurological disorder is epilepsy.

31. The method of claim 28, wherein the oligonucleotide is used in combination with another drug.
